(19) United States
(12) Patent Application Publication
(10) Pub. No.: US 2016/0305959 A1
(43) Pub. Date: Oct. 20, 2016

(54) DETECTION OF ATHEROSCLEROTIC CARDIOVASCULAR DISEASE RISK

(71) Applicants: THE UNITED STATES OF AMERICA, as represented by THE SECRETARY, DEPARTEMENT OF HEALTH AND HUMAN, Bethesda, MD (US); TRUSTEES OF BOSTON UNIVERSITY, Boston, MA (US)

(72) Inventors: Daniel Levy, Newton, MA (US); Martin G. Larson, Newton, MA (US)

(73) Assignees: THE UNITED STATES OF AMERICA, as represented by THE SECRETARY, DEPARTEMENT OF HEALTH AND HUMAN; HEALTH AND HUMAN SERVICES, Bethesda, MD (US); Trustees of Boston University, Boston, MD (US)

(21) Appl. No.: 15/036,751

(22) PCT Filed: Nov. 13, 2014
(86) PCT No.: PCT/US2014/065527

§ 371 Date: May 13, 2016

Related U.S. Application Data
(60) Provisional application No. 61/904,408, filed on Nov. 14, 2013.

(51) Int. Cl. G01N 33/68; C12Q 1/68

(52) U.S. Cl. CPC G01N 33/6893; C12Q 1/6883; G01N 2800/50; G01N 2800/52; G01N 2333/4728; G01N 2800/324; C12Q 2600/158; C12Q 2600/118; C12Q 2600/106; G01N 2560/00

(57) ABSTRACT

Methods are disclosed herein for determining the likelihood that a subject will develop atherosclerotic cardiovascular disease (ASCVD) or a myocardial infarction (MI). Methods are also disclosed for determining if a pharmaceutical agent is effective for the treatment or prevention of ASCVD. In additional embodiments, methods are disclosed for determining if a pharmaceutical agent is of use in preventing an MI.

### CROSS REFERENCE TO RELATED APPLICATIONS

This application claims priority to U.S. Provisional Application No. 61/904,408, filed Nov. 14, 2013, which is incorporated by reference in its entirety.

### FIELD OF THE DISCLOSURE

This relates to the field of cardiovascular disease, specifically to methods for determining if a subject is at risk for developing atherosclerosis or myocardial infarction.

### PARTIES TO JOINT RESEARCH AGREEMENT

The National Institutes of Health and Boston University are parties to a joint research agreement.

### BACKGROUND

Atherosclerotic cardiovascular disease (ASCVD) is the leading cause of death in the U.S. and is becoming the leading killer in developing countries. The lifetime risk of developing coronary heart disease (CHD) is 1 in 2 for men and 1 in 3 for women. In 2009, CVD ranked highest among all disease categories in hospital discharges; 16 million people in the US have CHD and 7 million have a history of stroke. Each year, an estimated 785,000 Americans will have a first CHD event and 610,000 a first stroke. Between 2010 and 2030, total direct medical costs of CVD are projected to triple, from $273 billion to $818 billion. There is a need to identify subjects with ASCVD, such as subjects with subclinical ASCVD, and those who are at risk for a myocardial infarction. In addition, there is a need to develop methods for diagnosis of subject with a myocardial infarction (MI), and methods to determine if a treatment regimen is effective.

### SUMMARY OF THE DISCLOSURE

Methods are disclosed for detecting or determining the likelihood that a subject will develop ASCVD. In some embodiments, the methods include performing one or more assays that detect the level of CD5-antigen like (CD5L), tetranectin (CLEC3B), and paraoxonasel (PON1) in a biological sample from the subject, and comparing the level of CD5L, CLEC3B, and PON1 to a respective control level of CD5L, CLEC3B, and PON1. The detection of a decrease in the level of CD5L, CLEC3B, and PON1 as compared to the respective control indicates that the subject has or will develop ASCVD. In some embodiments the method includes performing an assay that detects the level of alpha-1-acid glycoprotein 1 (ORM1) in a biological sample from the subject and comparing the level of ORM1 to a respective control level of ORM1. Detection of an increase in the level of ORM1 as compared to the control level of ORM1 indicates that the subject has or will develop ASCVD. In certain embodiments, the level of protein and/or mRNA can be detected.

Methods are also disclosed for determining the likelihood that a pharmaceutical agent is effective for treatment or prevention of ASCVD. In some embodiments, the methods include performing one or more assays that detect the level of CD5L, CLEC3B, and PON1 in a biological sample from the subject administered the agent, and comparing the level of CD5L, CLEC3B, and PON1 to a respective control level of CD5L, CLEC3B, and PON1. Detection of an increase in the level of CD5L, CLEC3B, and PON1 as compared to the respective control indicates that the pharmaceutical agent is effective for the treatment or prevention of atherosclerotic cardiovascular disease in the subject. In some embodiments, the methods also include performing an assay that detects the level of ORM1 in a biological sample from the subject, and comparing the level of ORM1 to a respective control level of ORM1. Detection of a decrease in the level of ORM1 as compared to the respective control level of ORM1 indicates that the pharmaceutical agent is effective for the treatment or prevention of atherosclerotic cardiovascular disease in the subject. In certain embodiments, the level of protein and/or mRNA can be detected.

Additional methods are disclosed for detecting or determining the likelihood that a subject will develop a myocardial infarction (MI). In some embodiments, these methods include performing one or more assays that detect the level of CDSL and C-reactive protein (CRP) in a biological sample from the subject, and comparing the level of CDSL and CRP to a respective control level of CDSL and CRP. Detection of a decrease in the level of CDSL and an increase in the level of CRP as compared to the respective control indicates that the subject will have an MI. In further embodiments, the methods include performing an assay that detects the level of protein Z-dependent protease inhibitor (SERPINA10) in a biological sample from the subject; and comparing the level of SERPINA10 to a respective control level of SERPINA10. The detection of an increase in the level of SERPINA10 as compared to the respective control level of SERPINA10 indicates that the subject has or will develop an MI. In certain embodiments, the level of protein and/or mRNA can be detected.

In further embodiments, methods are provided for determining the likelihood that a pharmaceutical agent is effective for prevention of an MI in a subject. The methods can include performing one or more assays that detect the level of CD5L and CRP in a biological sample from the subject administered the agent, and comparing the level of CD5L and CRP to a respective control level of CD5L and CRP. Detection of an increase in the level of CD5L and a decrease in the level of CRP as compared to the respective control indicates that the pharmaceutical agent is effective for prevention of the MI in the subject. In yet other embodiments, the methods include performing an assay that detects the level of SERPINA10 in a biological sample from the subject, and comparing the level of SERPINA10 to a respective control level of SERPINA10. Detection of a decrease in the level of SERPINA10 as compared to the respective control level of SERPINA10 indicates that the pharmaceutical agent is effective for the prevention of the MI. In certain embodiments, the level of protein and/or mRNA can be detected.

In some embodiments, methods are provided for determining the likelihood that a subject will develop an MI. The methods can include performing an assay that detects the level of multimerin-2 (MMRN2) in a biological sample from the subject and comparing the level of MMRN2 to a respective control level of MMRN2. The detection of an increase in the level of MMRN2 as compared to the control indicates that the subject will have an MI. These methods can also include performing one or more assays that detect the level of CD5 antigen like (CD5L), cyclophilin A (PPIA), C-reactive protein (CRP), collagen alpha-1(XVIII) chain (COL18A1), alpha-amylase 1 (salivary) (AMY1A), and cell surface glycoprotein MUC18 (MCAM) in a biological sample from the subject, and comparing the level of CD5L, PPIA, CRP, COL18A1, AMY1A, and MCAM to a respective control level of CD5L, PPIA, CRP, COL18A1, AMY1A, and MCAM. The detection of a decrease in the level of CD5L, PPIA, AMY1A, and MCAM and an increase in the level of CRP and COL18A1, as compared to the respective control level of CD5L, PPIA, CRP, COL18A1, AMY1A, and MCAM indicates that the subject has or will develop an MI. In certain embodiments, the level of protein and/or mRNA can be detected.

In yet other embodiments, methods are provided for determining a pharmaceutical agent is effective for prevention of an MI in a subject. The methods include performing an assay that detects the level of MMRN2 in a biological sample from the subject and comparing the level of MMRN2 to a respective control level of MMRN2. Detection of a decrease in the level MMRN2 as compared to the control indicates that the pharmaceutical agent is effective for prevention of the MI. These methods can also include performing one or more assays that detect the level of CD5L, PPIA, CRP, COL18A1, AMY1A, and MCAM in a biological sample from the subject and comparing the level of CD5L, PPIA, CRP, COL18A1, AMY1A and MCAM to a respective control level of CD5L, PPIA, CRP, COL18A1, AMY1A and MCAM. The detection of an increase in the level of CD5L, PPIA, AMY1A, and MCAM and a decrease in the level of CRP and COL18A1, as compared to the respective control level of CD5L, PPIA, CRP, COL18A1, AMY1A, and MCAM indicates that the pharmaceutical agent is effective for the treatment or prevention of the MI in the subject. In certain embodiments, the level of protein and/or mRNA can be detected.

The foregoing and other features and advantages of the invention will become more apparent from the following detailed description of a several embodiments which proceeds with reference to the accompanying figures.

### BRIEF DESCRIPTION OF THE FIGURES

FIG. 1. Flow chart of experimental design using discovery and targeted mass spectrometry.

### SEQUENCES

GENBANK® Accession numbers are provided below. In these entries nucleic and amino acid sequences listed are shown using standard letter abbreviations for nucleotide bases, and one letter code for amino acids, as defined in 37 C.F.R. 1.822. Only one strand of each nucleic acid sequence is shown, but the complementary strand is understood as included by any reference to the displayed strand. All of the GENBARNK® entries are incorporated herein by reference as available on Nov. 1, 2013. Each GENBANK® Accession number listed herein entry provides an exemplary sequence for the listed proteins. Polypeptides and polynucleotides at least 95%, 96%, 97%, 98% or 99% identical to the polypeptide or nucleic acid sequence shown in these GENBANK® entries, respectively, and be of use in the methods disclosed herein.

### DETAILED DESCRIPTION OF SEVERAL EMBODIMENTS

The current methods of ASCVD and MI risk assessment are suboptimal. Many people are misclassified with regard to high, intermediate, or low risk groups. Misclassification of risk results in incorrect treatment decisions and worse outcomes. Better risk assessment will reduce risk misclassification and improve treatment decisions and outcomes.

It is disclosed herein that mass spectrometry (MS)-based (iTRAQ™ isotopic labeling) and MRM mass spectrometry were used to identify differentially expressed proteins that can be used to determine a subject's risk for ASCVD or MI, and can be used to evaluate the efficacy of a therapeutic protocol for the treatment of these conditions.

### Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. All GENBANK® Accession Nos. listed herein are incorporated by reference in their entirety. Although methods and materials similar or equivalent to those described herein can be used in the practice or testing of the present disclosure, suitable methods and materials are described below. The materials, methods, and examples are illustrative only and not intended to be limiting.

Alter: A change in an effective amount of a substance of interest, such as a polynucleotide or polypeptide. The amount of the substance can changed by a difference in the amount of the substance produced, by a difference in the amount of the substance that has a desired function, or by a difference in the activation of the substance. The change can be an increase or a decrease. The alteration can be in vivo or in vitro.

In several embodiments, altering an amount of a polypeptide or polynucleotide is at least about a 50%, 60%, 70%, 80%, 90%, 95%, 96%, 97%, 98%, 99%, or 100% increase or decrease in the effective amount (level) of a substance. In specific example, an increase of a polypeptide or polynucleotide is at least about a 50%, 60%, 70%, 80%, 90%, 95%, 96%, 97%, 98%, 99%, or 100% increase in a polypeptide or polynucleotide as compared to a control, a statistical normal, or a standard value chosen for specific study. In another specific example, a decrease of a polypeptide or polynucleotide, such as following the initiation of a therapeutic protocol, is at least about a 50%, 60%, 70%, 80%, 90%, 95%, 96%, 97%, 98%, 99%, or 100% decrease in a polypeptide or polynucleotide as compared to a control, a statistical normal, or a standard value chosen for specific study.

Antibody: A polypeptide including at least a light chain or heavy chain immunoglobulin variable region which specifically recognizes and binds an epitope of an antigen or an antigen-binding fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Antibodies of the present disclosure include those that are specific for the molecules listed.

The term antibody includes intact immunoglobulins, as well the variants and portions thereof, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA, and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.”

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “polyclonal antibody” is an antibody that is derived from different B-cell lines. Polyclonal antibodies are a mixture of immunoglobulin molecules secreted against a specific antigen, each recognizing a different epitope. These antibodies are produced by methods known to those of skill in the art, for instance, by injection of an antigen into a suitable mammal (such as a mouse, rabbit or goat) that induces the B-lymphocytes to produce IgG immunoglobulins specific for the antigen, which are then purified from the mammal's serum.

A “chimeric antibody” has framework residues from one species, such as human, and CDRs (which generally confer antigen binding) from another species, such as a murine antibody that specifically binds an antigen of interest.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one example, all the CDRs are from the donor immunoglobulin in a humanized immunoglobulin. Constant regions need not be present, but if they are, they are substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. Humanized immunoglobulins can be constructed by means of genetic engineering (see for example, U.S. Pat. No. 5,585,089).

Array: An arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called chips or biochips.

The array of molecules (“features”) makes it possible to carry out a very large number of analyses on a sample at one time. In certain example arrays, one or more molecules (such as an oligonucleotide probe) will occur on the array a plurality of times (such as twice), for instance to provide internal controls. The number of addressable locations on the array can vary, for example from at least one, to at least 2, to at least 5, to at least 10, at least 20, at least 30, at least 50, at least 75, at least 100, at least 150, at least 200, at least 300, at least 500, least 550, at least 600, at least 800, at least 1000, at least 10,000, or more. In particular examples, an array includes nucleic acid molecules, such as oligonucleotide sequences that are at least 15 nucleotides in length, such as about 15-40 nucleotides in length. In particular examples, an array includes oligonucleotide probes or primers which can be used to detect ASCVD and MI.

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Protein-based arrays include probe molecules that are or include proteins, or where the target molecules are or include proteins, and arrays including antibodies to which proteins are bound, or vice versa. In some examples, an array contains antibodies to ASCVD and MI-associated proteins.

In some examples, the array includes positive controls, negative controls, or both, for example molecules specific for detecting β-actin, 18S RNA, beta-microglobulin, glyceraldehyde-3-phosphate-dehydrogenase (GAPDH), and other housekeeping genes. In one example, the array includes 1 to 20 controls, such as 1 to 10 or 1 to 5 controls.

Atherosclerosis: The progressive narrowing and hardening of a blood vessel over time. Atherosclerosis is a common form of arteriosclerosis in which deposits of yellowish plaques (atheromas) containing cholesterol, lipoid material and lipophages are formed within the intima and inner media of large and medium-sized arteries. Treatment of atherosclerosis includes reversing or slowing the progression of atherosclerosis, for example as measured by the presence of atherosclerotic lesions and/or functional signs of the disease, such as improvement in cardiovascular function as measured by signs (such as peripheral capillary refill), symptoms (such as chest pain and intermittent claudication), or laboratory evidence (such as that obtained by EKG, angiography, or other imaging techniques). “Assessing atherosclerosis” indicates determining if a subject of interest has atherosclerosis, determining the prognosis of the subject of interest, and/or determining if a therapeutic regimen administered to the subject is effective in treating the subject.

Bile acid binding resins: Agents that lower LDL cholesterol. Bile acids are the breakdown products of cholesterol and are excreted by the liver via the bile. Bile acids are 90% reabsorbed from the intestine and used to re-manufacture cholesterol in the liver. Bile acid binding resins (also referred to as bind acid sequestrants) interfere with this intestinal reabsorption by binding bile acids in the gut and promoting their excretion from the body.

Blood vessel: The vessels through which blood circulates. In general, blood vessels are elastic tubular channels that are lined with endothelium. Blood vessels include the arteries, veins and capillaries. Specific, non-limiting examples of a blood vessel include a vena cava, a thoracic aorta, a saphenous vein, a mammary artery, the brachial artery and a capillary. In another embodiment, a blood vessel includes the smaller arteries and veins. In yet another embodiment, a blood vessel is a capillary of the microvascular circulation.

Cardiovascular: Pertaining to the heart and/or blood vessels.

Cardiovascular disease (CVD): Disorders of the heart and blood vessels, such as atherosclerosis (ASCVD), coronary heart disease, cerebrovascular disease, and peripheral vascular disease. Cardiovascular diseases also include, for example, myocardial infarction, stroke, angina pectoris, transient ischemic attacks, and congestive heart failure. Atherosclerosis usually results from the accumulation of fatty material, inflammatory cells, extracellular matrices and plaque. Clinical symptoms and signs indicating the presence of CVD may include one or more of the following: chest pain and other forms of angina, shortness of breath, sweatiness, Q waves or inverted T waves on an EKG, a high calcium score by CT scan, at least one stenotic lesion on coronary angiography, and heart attack. Subclinical ASCVD can be identified by imaging tests (such as CT measures of coronary calcification, or MRI measures of coronary or aortic plaque, and/or ultrasound evidence of carotid plaque or thickening).

Cardiovascular risk: The likelihood of the development of cardiovascular disease, such as, but not limited to, myocardial ischemia and infarction, intermittent claudication, transient ischemic attacks, ischemic strokes, and other conditions associated with cardiovascular dysfunction. In a specific non-limiting example, the disorder is atherosclerosis. Similarly, atherosclerotic risk is the likelihood of the development of atherosclerosis, myocardial infarction risk is the likelihood of having a myocardial infarction, and heart failure risk is the likelihood of developing heart failure.

Cholesterol absorption inhibitor: A class of cholesterol lowering drugs that block absorption of cholesterol at the brush border of the intestine without affecting absorption of triglycerides or fat soluble vitamins. These drugs are not systemically absorbed and can lower cholesterol on their own (i.e. without the use of additional drugs). An exemplary cholesterol absorption inhibitor is ezetimibe (Ezetrol).

Cholesterol lowering agent: An agent that lowers the level of cholesterol in a subject, such as a pharmaceutical, vitamin, or small molecule. One of skill in the art can readily identify assays, such as blood screening, to determine the effect of cholesterol. Agents include, but are not limited to, niacin, the statins (e.g., ZOCOR™, LIPITOR™, PRAVACOL™, LESCOR™ MEVACOR™), bile acid binding resins (e.g., QUESTRAN™), and fibrates (e.g. LOPID™, LIPIDIL MICRO™).

Consists essentially of: In the context of the present disclosure, “consists essentially of” indicates that the expression of additional markers associated with a disorder can be evaluated, but not more than ten additional associated markers. In some examples, “consists essentially of” indicates that no more than 5 other molecules are evaluated, such as no more than 4, 3, 2, or 1 other molecules. In some examples, the expression of one or more controls is evaluated, such as a housekeeping protein or rRNA (such as 18S RNA, beta-microglobulin, GAPDH, and/or β-actin) in addition to the genes associated with the disorder. In this context “consists of” indicates that only the expression of the stated molecules is evaluated; the expression of additional molecules is not evaluated.

Control: A “control” refers to a sample or standard used for comparison with an experimental sample. In some embodiments, the control is a sample obtained from a healthy patient or a non-diseased tissue sample obtained from a patient diagnosed with the disorder of interest, such as MI or ASCVD. In some embodiments, the control is a historical control or standard reference value or range of values (such as a previously tested control sample, such as a group of patients with the disorder, or group of samples that represent baseline or normal values, such as the level of specific genes in non-diseased tissue).

Detecting expression of a gene product: Determining the presence of and/or the level of expression of a nucleic acid molecule (such as an mRNA molecule) or a protein encoded by a gene in either a qualitative or quantitative manner. Exemplary methods include microarray analysis, RT-PCR, Northern blot, Western blot, and mass spectrometry of specimens from a subject, for example measuring levels of a gene product present in blood, serum, or another biological sample as a measure of expression.

Diagnosis: The process of identifying a disease by its signs, symptoms and results of various tests. The conclusion reached through that process is also called “a diagnosis.” Forms of testing commonly performed include blood tests, medical imaging, urinalysis, and biopsy.

Differential or alteration in expression: A difference or change, such as an increase or decrease, in the conversion of the information encoded in a gene into messenger RNA, the conversion of mRNA to a protein, or both. In some examples, the difference is relative to a control or reference value or range of values, such as an amount of gene expression that is expected in a subject who does not have a disorder of interest (for example heart disease, atherosclerosis or myocardial infarction). Detecting differential expression can include measuring a change in gene expression or a change in protein levels.

Downregulated or decreased: When used in reference to the expression of a nucleic acid molecule, such as a gene, refers to any process which results in a decrease in production of a gene product. A gene product can be RNA (such as microRNA, mRNA, rRNA, tRNA, and structural RNA) or protein. Therefore, gene downregulation or deactivation includes processes that decrease transcription of a gene or translation of mRNA.

Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene downregulation can include reduction of expression above an existing level. Examples of processes that decrease translation include those that decrease translational initiation, those that decrease translational elongation and those that decrease mRNA stability.

Gene downregulation includes any detectable decrease in the production of a gene product. In certain examples, production of a gene product decreases by at least 2-fold, for example at least 3-fold or at least 4-fold, as compared to a control (such an amount of gene expression in a normal cell). In one example, a control is a relative amount of gene expression in a biological sample, such as from a subject that does not have ASCVD or has not had an MI.

Expression: The process by which the coded information of a gene is converted into an operational, non-operational, or structural part of a cell, such as the synthesis of a protein. Gene expression can be influenced by external signals. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced. In an example, gene expression can be monitored to determine the diagnosis and/or prognosis of a subject with ASCVD or at risk for an MI.

The expression of a nucleic acid molecule in a test sample can be altered relative to a control sample, such as a normal sample from a healthy subject. Expression of proteins is the level of protein in a biological sample. Expression includes, but is not limited to, the production of the protein by translation of an mRNA and the half-life of the protein. Protein expression can also be altered in some manner to be different from the expression of the protein in a normal (e.g., non-disease) situation. Alterations in expression, such as differential expression, include but are not limited to: (1) overexpression; (2) underexpression; or (3) suppression of expression.

Controls or standards for comparison to a sample, for the determination of differential expression, include samples believed to be normal (in that they are not altered for the desired characteristic, for example a sample from a subject who does not have ASCVD) as well as laboratory values (e.g., range of values), even though possibly arbitrarily set, keeping in mind that such values can vary from laboratory to laboratory. Laboratory standards and values can be set based on a known or determined population value and can be supplied in the format of a graph or table that permits comparison of measured, experimentally determined values.

Fibrates: Agents that lower triglyceride levels and raise HDL levels. Fibrates, also known as fibric acid derivatives, are particularly useful in diabetic patients whose characteristic lipid abnormality is high triglycerides and low HDL. In some patients who have combined lipid abnormalities, fibrates are combined with statins to lower both triglycerides and LDL and to raise HDL. Exemplary fibrates include gemfibrozil (LOPID™), fenofibrate (Lipidil micro, Lipidil Supra, Lipidil EZ), and bezafibrate (Bezalip).

Framingham Risk Score: A risk factor score that is used for predicting future risk of coronary artery disease in individuals free of disease, based on the measurement of Framingham risk factors which include age, gender, systolic blood pressure (and use of antihypertensive treatment), cigarette smoking, diabetes, as well as total cholesterol (or low density lipoprotein cholesterol (LDL cholesterol) and high density lipoprotein cholesterol (HDL cholesterol) levels (Wilson et al., Circulation 1998; 97: 1837-47).

Gene expression profile (or signature): Differential or altered gene expression can be detected by changes in the detectable amount of gene expression (such as cDNA or mRNA) or by changes in the detectable amount of proteins expressed by those genes. A distinct or identifiable pattern of gene expression, for instance a pattern of high and low expression of a defined set of genes or gene-indicative nucleic acids such as ESTs. A gene expression profile (also referred to as a signature) can be linked to disease progression (such as advanced ASCVD), or to any other distinct or identifiable condition that influences gene expression in a predictable way. Gene expression profiles can include relative as well as absolute expression levels of specific genes, and can be viewed in the context of a test sample compared to a baseline or control sample profile (such as a sample from the same tissue type from a subject who does not have ASCVD). In one example, a gene expression profile in a subject is read on an array (such as a nucleic acid or protein array). For example, a gene expression profile can be performed using a commercially available array such as Human Genome GENECHIP® arrays from AFFYMETRIX® (Santa Clara, Calif.).

Hybridization: To form base pairs between complementary regions of two strands of DNA, RNA, or between DNA and RNA, thereby forming a duplex molecule, for example. Hybridization conditions resulting in particular degrees of stringency will vary depending upon the nature of the hybridization method and the composition and length of the hybridizing nucleic acid sequences. Generally, the temperature of hybridization and the ionic strength (such as the Na concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (detects sequences that share at least 90% identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (detects sequences that share at least 80% identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (detects sequences that share at least 60% identity)

Hybridization: 6×SSC at RT to 55° C. for 16-20 hours

Wash at least twice: 2×-3×SSC at RT to 55° C. for 20-30 minutes each

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins.

Label: An agent capable of detection, for example by ELISA, spectrophotometry, flow cytometry, or microscopy. For example, a label can be attached to a nucleic acid molecule or protein, thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998). In a particular example, a label is conjugated to a binding agent that specifically binds to an ASCVD and MI associated protein, disclosed herein.

Level of Expression: An amount, such as of a protein or an mRNA, that can be measured in a biological sample.

Lipoprotein: A biochemical assembly that contains both proteins and lipids, bound to the proteins, which allow fats to move through the water inside and outside cells. There are five major groups of lipoprotein particles, which, in order of molecular size, largest to smallest, are chylomicrons, very low-density lipoprotein (VLDL), intermediate-density lipoprotein (IDL), low-density lipoprotein (LDL), and HDL. HDL contains the highest proportion of protein to cholesterol; its most abundant apolipoproteins are apo A-I and apo A-II. LDL contains apolipoprotein B, and has a core consisting of linoleate and includes esterified and non-esterified cholesterol molecules. LDL particles are approximately 22 nm in diameter and have a mass of about 3 million daltons. Lipoprotein a is a lipoprotein subclass; lipoprotein a consists of an LDL-like particle and the specific apolipoprotein(a) [apo(a)], which is covalently bound to the apolipoprotein B of the LDL like particle.

Mammal: This term includes both human and non-human mammals. Examples of mammals include, but are not limited to: humans, pigs, cows, goats, cats, dogs, rabbits, rats, and mice.

Marker: A biological marker, such as a polypeptide or a polynucleotide, that can be detected in a biological sample from a subject. A polypeptide marker can be about 95%, about 96%, about 97%, about 98%, about 99% or 100% identical to a reference amino acid sequence, such as a known amino acid sequence provided in a database such as GENBANK® or EMBL®. In some embodiments, an antibody that specifically binds a reference sequence of interest will bind the marker in a biological sample. A polynucleotide marker can be about 95%, 96%, 97%, 98%, 99% or 100% identical to a reference nucleic acid sequence, such as a known nucleic acid sequence provided in a database such as GENBANK® or EMBL®. In some embodiments, a probe that specifically hybridizes to a reference sequence under very stringent conditions will bind the polynucleotide marker, or primers designed to amplify the reference sequence will amplify the polynucleotide marker.

Mass Spectrometry: A process used to separate and identify molecules based on their mass. Mass spectrometry ionizes chemical compounds to generate charged molecules or molecule fragments and measures their mass-to-charge ratios. In a typical MS procedure, as sample is ionized. The ions are separated according to their mass-to-charge ratio, and the ions are dynamically detected by some mechanism capable of detecting energetic charged particles. The signal is processed into the spectra of the masses of the particles of that sample. The elements or molecules are identified by correlating known masses by the identified masses. “Time-of-flight mass spectrometry” (TOFMS) is a method of mass spectrometry in which an ion's mass-to-charge ratio is determined via a time measurement. Ions are accelerated by an electric field of known strength. This acceleration results in an ion having the same kinetic energy as any other ion that has the same charge. The velocity of the ion depends on the mass-to-charge ratio. The time that it subsequently takes for the particle to reach a detector at a known distance is measured. This time will depend on the mass-to-charge ratio of the particle (heavier particles reach lower speeds). From this time and the known experimental parameters one can find the mass-to-charge ratio of the ion. “Liquid chromatography-mass spectrometry” or “LC-MS” is a chemistry technique that combines the physical separation capabilities of liquid chromatography (or HPLC) with the mass analysis capabilities of mass spectrometry. Liquid chromatography mass spectrometry (LC-MS) separates compounds chromatographically before they are introduced to the ion source and mass spectrometer. It differs from gas chromatography (GC-MS) in that the mobile phase is liquid, usually a mixture of water and organic solvents, instead of gas and the ions fragments. Most commonly, an electrospray ionization source is used in LC-MS.

Multiple reaction monitoring (MRM): A mass spectrometry based method in which absolute quantification of a targeted protein(s) can be obtained. In this method external or internal standards are used. Often a known quantity of a synthetic stable isotopically labeled peptide matching each of the targeted peptides that represent unique the protein is added into each sample being quantified. Comparison of the peak of the endogenous peptide to the labeled standard peptide allows absolute quantitation. MRM can be multiplexed easily, allowing multiple phosphorylation sites and/or multiple proteins to be assessed simultaneously.

Myocardial Infarction (MI): An event that occurs when blood stops flowing properly to part of the heart and the heart muscle is injured due to inadequate oxygen delivery. Acute myocardial infarction refers to two subtypes of acute coronary syndrome, namely non-ST-elevated myocardial infarction and ST-elevated myocardial infarction, which are most frequently (but not always) a manifestation of coronary artery disease. The most common triggering event is the disruption of an atherosclerotic plaque in an epicardial coronary artery, which leads to a clotting cascade, sometimes resulting in total occlusion of the artery. If impaired blood flow to the heart lasts long enough, it triggers a process called the ischemic cascade; the heart cells in the territory of the occluded coronary artery die, chiefly through necrosis. A collagen scar forms in the heart in place of the damaged cells.

Niacin: A B-vitamin that is used as a medication for patients with elevated levels of triglycerides and cholesterol. A long-acting preparation of niacin is available as NIASPAN®.

Nucleic acid array: An arrangement of nucleic acids (such as DNA or RNA) in assigned locations on a matrix, such as that found in cDNA arrays, or oligonucleotide arrays.

Nucleic acid molecules representing genes: Any nucleic acid, for example DNA (intron or exon or both), cDNA, or RNA (such as mRNA), of any length suitable for use as a probe or other indicator molecule, and that is informative about the corresponding gene, such the proteins specified herein.

PCSK9-targeting drug: An agent that targets proprotein convertase subtilisin/kexin type 9 (PCSK9), an enzyme that mediates the post-translational degradation of the LDL receptor, thereby modulating serum levels of LDL cholesterol. Thus, drugs that inhibit PCSK9 also lower cholesterol. A number of PCSK9-targeting drugs are in development, including monoclonal antibodies, small molecules and gene silencing agents (e.g., antisense oligonucleotides, locked nucleic acids, and siRNAs).

Peptide/Protein/Polypeptide: All of these terms refer to a polymer of amino acids and/or amino acid analogs that are joined by peptide bonds or peptide bond mimetics, regardless of length or post-translational modification (such as glycosylation, methylation, ubiquitination, phosphorylation, or the like).

Polymerase Chain Reaction (PCR): An in vitro amplification technique that increases the number of copies of a nucleic acid molecule (for example, a nucleic acid molecule in a sample or specimen). The product of a PCR can be characterized by standard techniques known in the art, such as electrophoresis, restriction endonuclease cleavage patterns, oligonucleotide hybridization or ligation, and/or nucleic acid sequencing.

In some examples, PCR utilizes primers, for example, DNA oligonucleotides 10-100 nucleotides in length, such as about 15, 20, 25, 30 or 50 nucleotides or more in length (such as primers that can be annealed to a complementary target DNA strand by nucleic acid hybridization to form a hybrid between the primer and the target DNA strand. Primers can be selected that include at least 15, at least 20, at least 25, at least 30, at least 35, at least 40, at least 45, at least 50 or more consecutive nucleotides of a nucleotide sequence of interest. Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990).

Primers: Short nucleic acid molecules, for instance DNA oligonucleotides 10-100 nucleotides in length, such as about 15, 20, 25, 30 or 50 nucleotides or more in length, such as this number of contiguous nucleotides of a nucleotide sequence encoding a protein of interest or other nucleic acid molecule. Primers can be annealed to a complementary target DNA strand by nucleic acid hybridization to form a hybrid between the primer and the target DNA strand. Primer pairs can be used for amplification of a nucleic acid sequence, such as by PCR or other nucleic acid amplification methods known in the art.

Methods for preparing and using nucleic acid primers are described, for example, in Sambrook et al. (In Molecular Cloning: A Laboratory Manual, CSHL, New York, 1989), Ausubel et al. (In Current Protocols in Molecular Biology, John Wiley & Sons, New York, 1998), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular primer increases with its length.

In one example, a primer includes at least 15 consecutive nucleotides of a nucleotide molecule, such as at least 18 consecutive nucleotides, at least 20, at least 25, at least 30, at least 35, at least 40, at least 45, at least 50 or more consecutive nucleotides of a nucleotide sequence (such as a gene, mRNA or cDNA). Such primers can be used to amplify a nucleotide sequence of interest, such as the markers listed in Tables A, B, and/or C, for example using PCR.

Probe: A short sequence of nucleotides, such as at least 8, at least 10, at least 15, at least 20, at least 21, at least 25, at least 30, at least 35, at least 40, at least 45, at least 50, at least 55, at least 60, at least 65, at least 70, at least 75, at least 80, at least 85, at least 90, at least 95 or even greater than 100 nucleotides in length, used to detect the presence of a complementary sequence by molecular hybridization. In particular examples, oligonucleotide probes include a label that permits detection of oligonucleotide probe:target sequence hybridization complexes. Such an oligonucleotide probe can also be used on a nucleic acid array, for example to detect a nucleic acid molecule in a biological sample contacted to the array. In some examples, a probe is used to detect the presence of a nucleic acid molecule for a markers listed in Tables A, B, and/or C.

Prognosis: A prediction of the future course of a disease, such as ASCVD. The prediction can include determining the likelihood of a subject to develop complications of ASCVD, such as an MI or a stroke, or to survive a particular amount of time (e.g., determine the likelihood that a subject will survive 1, 2, 3 or 5 years), to respond to a particular therapy (e.g., lipid lowering therapy), or combinations thereof.

Purified: The term “purified” does not require absolute purity; rather, it is intended as a relative term. Thus, for example, a purified protein preparation is one in which the protein referred to is more pure than the protein in its natural environment within a cell. For example, a preparation of a protein is purified such that the protein represents at least 50% of the total protein content of the preparation. Similarly, a purified oligonucleotide preparation is one in which the oligonucleotide is more pure than in an environment including a complex mixture of oligonucleotides.

Sample (or biological sample): A biological specimen containing genomic DNA, RNA (including mRNA), protein, or combinations thereof, obtained from a subject. Examples include, but are not limited to, peripheral blood, serum, plasma, urine, fine needle aspirate, tissue biopsy, surgical specimen, and autopsy material.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology (NCBI, National Library of Medicine, Building 38A, Room 8N805, Bethesda, Md. 20894) and on the Internet, for use in connection with the sequence analysis programs blastp, blastn, blastx, tblastn, and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs may use SEG filtering (Wootton and Federhen, Meth. Enzymol. 266:554-571, 1996). In addition, a manual alignment can be performed. Proteins with even greater similarity will show increasing percentage identities when assessed by this method, such as at least about 75%, 80%, 85%, 90%, 95%, 98%, or 99% sequence identity to a molecule listed in Tables A, B, or C.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity to a molecule listed in Tables A, B, or C. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity to a molecule listed in Tables A, B, or C determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Specific Binding Agent: An agent that binds substantially or preferentially only to a defined target such as a protein, enzyme, polysaccharide, oligonucleotide, DNA, RNA, recombinant vector or a small molecule. Thus, a nucleic acid-specific binding agent binds substantially only to the defined nucleic acid, such as RNA, or to a specific region within the nucleic acid. For example, a “specific binding agent” includes an antisense compound (such as an antisense oligonucleotide, siRNA, miRNA, shRNA or ribozyme) that binds substantially to a specified RNA.

A protein-specific binding agent binds substantially only the defined protein, or to a specific region within the protein. For example, a “specific binding agent” includes antibodies and other agents that bind substantially to a specified polypeptide. Antibodies can be monoclonal or polyclonal antibodies that are specific for the polypeptide, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

Statin: Any of a class of lipid-lowering drugs that reduce serum cholesterol levels by inhibiting a key enzyme involved in the biosynthesis of cholesterol. Example statins include atorvastatin (LIPITOR®), fluvastatin (LESCOL®), lovastatin (MEVACOR®, ALTOCOR®, not marketed in the UK), pravastatin (PRAVACHOL®, SELEKTINE®, LIPOSTAT®), rosuvastatin (CRESTOR®), simvastatin (ZOCOR®). There are two groups of statins: (1) Fermentation-derived: lovastatin, simvastatin and pravastatin, and (2) Synthetic statins: fluvastatin, atorvastatin, cerivastatin and rosuvastatin. Generally, statins act by competitively inhibiting 3-hydroxy-3-methylglutaryl coenzyme A (HMG CoA) reductase, an enzyme of the HMG-CoA reductase pathway, the body's metabolic pathway for the synthesis of cholesterol.

The structure of one exemplary statin, lovastatin, is shown below.

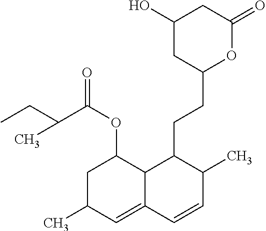 [SMILES: CCC(C)C(=O)OC1CC(C)C=C2C=CC(C)C(CCC3CC(O)CC(=O)O3)C21]

Subject: Living multi-cellular vertebrate organism, a category that includes human and non-human mammals.

Therapeutically effective amount: An amount of a pharmaceutical preparation that alone, or together with a pharmaceutically acceptable carrier or one or more additional therapeutic agents, induces the desired response. A therapeutic agent, such as an anticoagulant, or a statin, is administered in therapeutically effective amounts.

Effective amounts a therapeutic agent can be determined in many different ways, such as assaying for a reduction in atherosclerotic disease or improvement of physiological condition of a subject having vascular disease. Effective amounts also can be determined through various in vitro, in vivo or in situ assays.

Therapeutic agents can be administered in a single dose, or in several doses, for example daily, during a course of treatment. However, the effective amount of can be dependent on the source applied, the subject being treated, the severity and type of the condition being treated, and the manner of administration.

In one example, it is an amount sufficient to partially or completely alleviate symptoms of vascular disease within a subject. Treatment can involve only slowing the progression of the vascular disease temporarily, but can also include halting or reversing the progression of the vascular disease permanently. For example, a pharmaceutical preparation can decrease one or more symptoms of vascular disease, for example decrease a symptom by at least 20%, at least 50%, at least 70%, at least 90%, at least 98%, or even at least 100%, as compared to an amount in the absence of the pharmaceutical preparation.

Translation: The process in which cellular ribosomes create proteins. In translation, messenger RNA (mRNA) produced by transcription is decoded by a ribosome complex to produce a specific polypeptide.

Treating a disease: “Treatment” refers to a therapeutic intervention that ameliorates a sign or symptom of a disease or pathological condition, such a sign, parameter or symptom of vascular disease (for example, ASCVD). Treatment can also induce remission or cure of a condition, such as vascular disease. In particular examples, treatment includes preventing a disease, for example by inhibiting the full development of a disease, such as preventing development of vascular disease. Prevention of a disease does not require a total absence of vascular disease. For example, a decrease of at least 50% can be sufficient.

Upregulated or activation: When used in reference to the expression of a nucleic acid molecule, such as a gene, refers to any process which results in an increase in production of a gene product. A gene product can be RNA (such as mRNA, rRNA, tRNA, and structural RNA) or protein. Therefore, gene upregulation or activation includes processes that increase transcription of a gene or translation of mRNA.

Examples of processes that increase transcription include those that facilitate formation of a transcription initiation complex, those that increase transcription initiation rate, those that increase transcription elongation rate, those that increase processivity of transcription and those that relieve transcriptional repression (for example by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product. In certain examples, production of a gene product increases by at least 1.5-fold, such as at least 2-fold, at least 3-fold or at least 4-fold, as compared to a control. In one example, a control is a relative amount of gene expression in a biological sample, such as from a subject that does not have ASCVD or has not had an MI.

Additional terms commonly used in molecular genetics can be found in Benjamin Lewin, Genes V, published by Oxford University Press, 1994 (ISBN 0-19-854287-9); Kendrew et al (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

### Atherosclerotic Cardiovascular Disease (ASCVD) Risk

Methods are provided herein for evaluating cardiovascular risk, for example for determining the likelihood that a subject, such as an otherwise healthy subject, or a subject suspected or at risk of having cardiovascular disease, has cardiovascular disease or will likely develop cardiovascular disease, such as ASCVD in the future. In particular examples, the method can determine if a subject has or will likely develop ASCVD in the future. In further examples, the method can determine the likelihood that a pharmaceutical agent is effective for treating a subject.

In some examples, a biological sample obtained from the subject, such as, but not limited to, serum, blood, plasma, urine, purified cells (for example, blood cells, such as white blood cells, B cells, T cells, or mononuclear cells), saliva, a biopsy or tissue sample, such as a sample including blood vessels, adipose cells, or heart tissue obtained from the subject are used to predict the subject's risk of vascular disease.

In some embodiments, the subject is apparently healthy, such as a subject who does not exhibit symptoms of vascular disease (for example, does not have ASCVD, and/or has not previously had an acute adverse vascular event such as a myocardial infarction or a stroke). In some examples, a healthy subject is one that if examined by a medical professional, would be characterized as healthy and free of symptoms of vascular disease, such as ASCVD. The methods disclosed herein can be used to screen subjects for future evaluation or treatment for cardiovascular disease.

In other embodiments, the methods determine the likelihood that a subject will develop ASCVD. In specific non-liming examples, the subject is suspected of having a vascular disease, or is suspected of being at risk of developing a vascular disease, such as ASCVD in the future. For example, such a subject may have elevated cholesterol or tri-glyceride levels, elevated C-reactive protein levels, diabetes, or high blood pressure. The methods disclosed herein can be used to confirm a prior clinical suspicion of disease.

The expression of the markers disclosed herein can be used to assess the efficacy of a therapeutic protocol for the treatment or prevention of ASCVD. In some embodiments, methods are provided for evaluating the efficacy of a treatment protocol that includes any therapy for atherosclerosis designed to reverse or slow the progression of atherosclerosis, including but not limited to treatment with statins, niacin or other cholesterol-lowering agents, anti-inflammatory agents, aspirin, anti-platelet agents, anticoagulant agents, blood pressure lowering medications, agents for smoking cessation, or any other pharmaceutical compound.

In these embodiments, a sample can be taken from a subject prior to initiation of therapy. After therapy is initiated, an additional sample is taken from the subject. A decrease in the amount of the markers indicates that the therapy is efficacious. In addition, the subject can be monitored over time to evaluate the continued effectiveness of the therapeutic protocol. The effect of different dosages can also be evaluated, by comparing the expression of markers in a sample from the subject receiving a first dose to the expression of the same markers in a sample from the subject receiving a second (different) dose. The methods can be repeated 1, 2, 3, 4, 5, 6, 7, 8, 9, 10 or more times to determine the lowest dose of a pharmaceutical agent that is effective for treating the subject, and/or the shortest duration of administration that is effective for treating the subject. The methods can also be used over the course of a therapeutic regimen to monitor the efficacy of a pharmaceutical agent for the treatment of the subject.

In yet other embodiments, the subject has been determined to be at risk for cardiovascular disease based on risk factors, such as, but not limited to, Framingham risk factors. The Framingham Risk Score is a gender-specific algorithm used to estimate the 10-year cardiovascular risk of a subject using specific factors. The Framingham Risk Score was first developed based on data obtained from the Framingham Heart Study, to estimate the 10-year risk of developing coronary heart disease (see Third Report of the National Cholesterol Education Program (NCEP) Expert Panel on Detection, Evaluation, and Treatment of High Blood Cholesterol in Adults (Adult Treatment Panel III) final report, Circulation 2002 Dec. 17; 106(25):3143-421, incorporated herein by reference). The method can include evaluation of a subject to determine if they are at risk for cardiovascular disease using risk factors, such as, but not limited to, Framingham risk factors and/or guidelines jointly issues by the American Heart Association and American College of Cardiology.

Framingham risk factors include age, gender, low density lipoprotein (LDL) cholesterol level, whether the subject smokes, blood pressure (and whether the subject is receiving pharmacological treatment for hypertension), total cholesterol level, and high density lipoprotein (HDL) cholesterol level. Programs for this evaluation are available on the internet, such as at the U.S. National Heart, Lung, and Blood Institute (NHLBI) website.

In additional embodiments, the method does not comprise measuring a lipoprotein, such as LDL or HDL. In further embodiments, the method does not comprise measuring HDL or determining a HDL subpopulation based on size and/or density of the subject's HDL. In yet other embodiments, the method does not comprise measuring inflammatory markers. In specific, non-liming examples, the inflammatory marker is C reactive protein.

In some embodiments, the methods disclosed herein can include evaluating the level of one or more of the following:

TABLE A
Markers of Atherosclerosis

| HGNC Gene | UniProt/Swiss-Prot Full Name of | GENBANK |
|---|---|---|
| SYMBOL | Protein | Accession No. |
| ORM1 | Alpha-1-acid glycoprotein 1 | NM_000607.2 |
| PON1 | Paraoxonase/arylesterase 1 | NM_000446.5 |
| CLEC3B | Tetranectin | NM_003278.2 |
| CD5L | CD5 antigen-like | NM_005894.2 |

(All GENBANK ® Accession nucleic acid and amino acid sequences Incorporated by Reference as available on Nov. 1, 2013.)

The marker can include a polypeptide or nucleic acid sequence at least 95%, 96%, 97%, 98% or 99% identical to the polypeptide or nucleic acid sequence shown in these GENBANK® entries, respectively, or can be 100% identical to the listed sequence.

The methods can include assessing expression of 1, 2, 3, or all 4 of the markers listed in Table A in any combination. The methods can include evaluating the expression of one or more of the markers listed in Table 6. Proteins and mRNA can be evaluated, such as the level of 1, 2, 3, or all 4 of the markers shown in Table A.

In some embodiments, methods are provided for detecting or determining the likelihood that a subject will develop atherosclerotic cardiovascular disease. The methods can include performing one or more assays that detects an expression of CD5L, CLEC3B, and PON1 in a biological sample from the subject, and comparing the level of expression of CD5L, CLEC3B, and PON1 to a respective control level of CD5L, CLEC3B, and PON1. A decrease in expression level of CD5L, CLEC3B, and PON1 as compared to the respective control indicates that the subject has or will develop atherosclerotic cardiovascular disease. In some embodiments, the method also includes performing an assay that detects expression of ORM1 in a biological sample from the subject; and comparing the level of expression of ORM1 to a respective control level of ORM1. The detection of an increase in expression of ORM1 as compared to the respective control level of ORM1 indicates that the subject has or will develop atherosclerotic cardiovascular disease. The control can be a standard value of CD5L, CLEC3B, PON1 and/or ORM1, respectively or can be the level of CD5L, CLEC3B, PON1 and/or ORM1, respectively, in one or more subjects known not to have atherosclerotic cardiovascular disease. Proteins and/or mRNA can be evaluated.

Methods are also provided for determining if a pharmaceutical agent is effective for treatment or prevention of ASCVD in a subject. In specific non-liming examples, the subject can have ASCVD. In other embodiments, the subject has intermittent claudication, transient ischemic attacks (TIAs), ischemic strokes, restenosis after angioplasty, transplant atherosclerosis, unstable angina, or another condition associated with ASCVD.

The methods can include assessing expression of 1, 2, 3, or all 4 of the markers listed in Table A in any combination. In some embodiments, the methods include performing one or more assays that detect expression of CD5L, CLEC3B, and PON1 in a biological sample from the subject administered the agent; and comparing the level of expression of CD5L, CLEC3B, and PON1 to a respective control level of CD5L, CLEC3B, and PON1. The detection of an increase in expression of CD5L, CLEC3B, and PON1 as compared to the respective control indicates that the pharmaceutical agent is effective for the treatment or prevention of atherosclerotic cardiovascular disease in the subject. In additional embodiments, the method includes performing an assay that detects expression of ORM1 in a biological sample from the subject; and comparing the level of expression of ORM1 to a respective control level of ORM1. The detection of a decrease in expression of ORM1 as compared to the respective control level of ORM1 indicates that the pharmaceutical agent is effective for the treatment or prevention of atherosclerotic cardiovascular disease in the subject. In additional embodiments, the methods include assessing all of the markers listed in Table A. Proteins and or mRNA can be evaluated.

### Myocardial Infarction (MI) Risk

Methods are provided herein for evaluating the risk that a subject will have an MI, for example for determining whether a subject, such as an otherwise healthy subject, or a subject suspected or at risk of having cardiovascular disease, is likely have an MI in the future. In some examples, a biological sample obtained from the subject, such as, but not limited to, serum, blood, plasma, urine, purified cells (for example, blood cells, such as white blood cells, B cells, T cells, or mononuclear cells), saliva, a biopsy or tissue sample, such as a sample including blood vessels, adipose cells, or heart tissue obtained from the subject are used to predict the subject's risk of MI. The method disclosed herein can also be used to determine if a pharmaceutical agent is of use to delay or prevent an MI in the subject.

In some examples, a biological sample obtained from the subject, such as, but not limited to, serum, blood, plasma, urine, purified cells (for example, blood cells, such as white blood cells, B cells, T cells, or mononuclear cells), saliva, a biopsy or tissue sample, such as a sample including adipose cells, blood vessels or heart tissue obtained from the subject are used to predict the subject's risk of vascular disease.

In some embodiments, the subject is apparently healthy, such as a subject who does not exhibit symptoms of vascular disease (for example, does not have ASCVD, and/or has not previously had an acute adverse vascular event such as a previous MI or a stroke). In some examples, a healthy subject is one that if examined by a medical professional, would be characterized as healthy and free of symptoms of vascular disease, such as ASCVD. In some embodiments, a subject does not have a history of MI or other clinically apparent disease features). In further embodiments, the subject can have subclinical ASCVD identified by imaging tests (e.g., CT measures of coronary calcification, or MRI measures of coronary or aortic plaque, or ultrasound evidence of carotid plaque or thickening).

In other embodiments, the subject is suspected of having a vascular disease, or is suspected of being at risk of developing a vascular disease, and of being likely to have an MI the future. For example, such a subject may have elevated cholesterol or tri-glyceride levels, diabetes or high blood pressure. In other embodiments, the subject has intermittent claudication, bowel ischemia, retinal ischemia, transient ischemic attacks (TIAs), ischemic strokes, restenosis after angioplasty, transplant atherosclerosis, unstable angina, or another condition associated with cardiovascular dysfunction. Such a subject may also be without symptoms of ASCVD, but have evidence of subclinical CVD by virtue of medical testing, such as CT evidence of coronary calcification, MR evidence of coronary or aortic plaque, or ultrasound evidence of carotid plaque or thickening.

In yet other embodiments, the subject has been determined to be at risk for cardiovascular disease based on the aggregate burden of Framingham risk factors. The method can include evaluating a subject to determine if the subject is at risk for cardiovascular disease using Framingham risk factors. These risk factors include age, gender, whether the subject smokes, whether the subject has diabetes, blood pressure (and the use of medication to treat hypertension) total cholesterol level, and high density lipoprotein (HDL) cholesterol level (see above).

In additional embodiments, the method does not comprise measuring lipoprotein. In further embodiments, the method does not comprise measuring high density lipoproteins or determining a high density lipoprotein subpopulation.

Thus, the expression of the markers disclose herein can be used to assess the efficacy of a therapeutic protocol for the prevention of an MI. In some embodiments, methods are provided for evaluating the efficacy of a treatment protocol that includes any therapy for atherosclerosis designed to reverse or slow the progression of atherosclerosis, including but not limited to treatment with statins, niacin or other cholesterol-lowering agents. In additional embodiments, the treatment protocol includes the use of an antiplatelet agent, anticoagulation agent, lipid or blood pressure regulating agent. The agent can be a PCSK9-targeting drug, a bile acid binding resin, a LDL or HDL-cholesterol targeting drug, fish oils and/or omega-3 fatty acids. In some embodiments, the treatment protocol includes the use of a lipid regulating agent such as a statin, and doses are administered to lower LDL to 100 or below in a subject who is determined to have increased likelihood of myocardial infarction. In cases of very high risk an LDL of 70 or below may be targeted. In yet other embodiments, method are provided for identifying the amount of a therapeutic agent for administration. For example, high dose of a statin can be administered to a subject with high risk, while a lower dose of a statin can be administered in a subject with less risk.

In these embodiments, a sample can be taken from a subject prior to initiation of therapy. After therapy is initiated, an additional sample is taken from the subject. A decrease in the amount of the markers indicates that the therapy is efficacious. In addition, the subject can be monitored over time to evaluate the continued effectiveness of the therapeutic protocol. The effect of different dosages can also be evaluated, by comparing the expression of markers in a sample from the subject receiving a first dose to the expression of the same markers in a sample from the subject receiving a second (different) dose. The methods can be repeated 1, 2, 3, 4, 5, 6, 7, 8, 9, 10 or more times to determine the lowest dose of a pharmaceutical agent that is effective for treating the subject, and/or the shortest duration of administration that is effective for treating the subject. The methods can also be used over the course of a therapeutic regimen to monitor the efficacy of a pharmaceutical agent for the treatment of the subject.

In some embodiments, the method can include evaluating the level of one or more of the following:

TABLE B
Markers of MI

| HGNC Gene | UniProt/Swiss-Prot Full | GENBANK |
|---|---|---|
| SYMBOL | Name of Protein | Accession No. |
| SERPINA10 | Protein Z-dependent protease | AF181467.1 |
| | inhibitor | |
| CRP | C-reactive protein | NM_000567.2 |
| CD5L | CD5 antigen-like | NM_005894.2 |

(All GENBANK ® Accession nucleic acid and amino acid sequences Incorporated by Reference as available on Nov. 1, 2013.)

The marker can include a polypeptide or nucleic acid sequence at least 95%, 96%, 97%, 98% or 99% identical to the polypeptide or nucleic acid sequence shown in these GENBANK® entries, respectively, or can be 100% identical to the listed sequence.

The methods can include assessing expression of 1, 2, or all 3 of the markers listed in Table B. In other embodiments, the method includes assessing the expression of CRP in combination with one or both of SERPINA10 and CD5L. The methods can also include assessing the expression of one or more of the markers listed in Table 7. The methods can evaluate proteins and/or mRNA.

The methods can include performing one or more assays that detect expression of CD5L and CRP in a biological sample from the subject; and comparing the level of expression of CD5L and CRP to a respective control level of CD5L and CRP. The detection of a decrease in expression of CD5L and an increase in expression of CRP as compared to the respective control indicates that the subject will have a myocardial infarction. The method can evaluate the expression of protein and/or mRNA.

In some embodiments, the method also includes performing an assay that detects expression of SERPINA10 in a biological sample from the subject; and comparing the level of expression of SERPINA10 to a respective control level of SERPINA10. The detection of an increase in expression of SERPINA10 as compared to the respective control level of SERPINA10 indicates that the subject will develop a myocardial infarction. The method can evaluate the expression of protein and/or mRNA, such as by determining the level of protein and/or mRNA.

Methods are also provided for determining if a pharmaceutical agent is effective for prevention of MI in a subject. The methods can include assessing expression of 1, 2, or all 3 of the markers listed in Table B. In some embodiments, the methods include performing one or more assays that detect expression of CD5L and CRP in a biological sample from the subject administered the agent and comparing the level of expression of CD5L and CRP to a respective control level of CD5L and CRP. The detection of an increase in expression of CD5L and a decrease in expression of CRP as compared to the respective control indicates that the pharmaceutical agent is effective for prevention of the myocardial infarction in the subject. In additional embodiments, the method includes performing an assay that detects expression of SERPINA10 in a biological sample from the subject; and comparing the level of expression of SERPINA10 to a respective control level of SERPINA10. The detection of a decrease in expression of SERPINA10 as compared to the respective control level of SERPINA10 indicates that the pharmaceutical agent is effective for the prevention of an MI in the subject.

In further embodiments, the method can include evaluating the level of one or more of the following:

TABLE C
Markers of MI

| HGNC Gene | UniProt/Swiss-Prot Full | GENBANK |
|---|---|---|
| SYMBOL | Name of Protein | Accession No. |
| PPIA | Cyclophilin A | NM_021130.3 |
| CD5L | CD5 antigen-like | NM_005894.2 |
| MCAM | Cell surface glycoprotein MUC18 | M28882.1 |
| COL18A1 | Collagen alpha-1(XVIII) chain | BC063833.1 |
| AMY1A | Alpha-amylase 1 (salivary) | NM_004038 |
| CRP | C-reactive protein | NM_000567.2 |
| MMRN2 | Multimerin-2 | BC094744.1 |

(All GENBANK ® Accession nucleic acid and amino acid sequences Incorporated by Reference as available on Nov. 1, 2013.)

The marker can include a polypeptide or nucleic acid sequence at least 95%, 96%, 97%, 98% or 99% identical to the polypeptide or nucleic acid sequence shown in these GENBANK® entries, respectively, or can be 100% identical to the listed sequence.

The methods can include assessing expression of 1, 2, 3, 4, 5, 6, or all 7 of the markers listed in Table C. The methods can also include assessing the expression of one or more of the markers listed in Table 7. The methods can evaluate proteins and/or mRNA, such as the level of protein and/or mRNA for the 1, 2, 3, 4, 5, 6, or all 7 of the markers listed in Table C, or any 1, 2, 3, 4, 5 or 6 of the markers in combination with CRP.

In some embodiments, methods are provided for determining the likelihood that a subject will develop an MI. The methods include performing an assay that detects expression of MMRN2 in a biological sample from the subject and comparing the level of expression of MMRN2 to a respective control level of MMRN2. The detection of an increase in expression of MMRN2 as compared to the control indicates that the subject will have an Mi. These methods can also include performing one or more assays that detect expression of CD5L, PPIA, CRP, COL18A1, AMY1A, and MCAM in a biological sample from the subject, and comparing the level of expression of CD5L, PPIA, CRP, COL18A1, AMY1A and MCAM to a respective control level of CD5L, PPIA, CRP, COL18A1, AMY1A and MCAM. The detection of a decrease in expression of CD5L, PPIA, AMY1A, and MCAM and an increase in expression of CRP and COL18A1, as compared to the respective control level of CD5L, PPIA, CRP, COL18A1, AMY1A, and MCAM indicates that the subject has or will develop an MI.

In yet other embodiments, methods are provided for determining if a pharmaceutical agent is effective for prevention of an MI in a subject. The methods include performing an assay that detects expression of MMRN2 in a biological sample from the subject and comparing the level of expression of MMRN2 to a respective control level of MMRN2. Detection of a decrease in expression of MMRN2 as compared to the control indicates that the pharmaceutical agent is effective for prevention of the MI. These methods can also include performing one or more assays that detect expression of CD5L, PPIA, CRP, COL18A1, AMY1A, and MCAM in a biological sample from the subject and comparing the level of expression of CD5L, PPIA, CRP, COL18A1, AMY1A, and MCAM to a respective control level of CD5L, PPIA, CRP, COL18A1, AMY1A, and MCAM. The detection of an increase in expression of CD5L, PPIA, AMY1A, and MCAM and a decrease in expression of CRP and COL18A1, as compared to the respective control level of CD5L, PPIA, CRP, COL18A1, AMY1A, and MCAM indicates that the pharmaceutical agent is effective for the treatment or prevention of the MI in the subject.

### Methods for Detection of Proteins

In some examples, the level of expression of one or more proteins is analyzed by detecting and quantifying the protein in a biological sample. In particular examples, one or more proteins corresponding to the markers listed in Tables A, B, and C are analyzed. Suitable biological samples include samples containing protein, such as blood, serum, plasma, urine, saliva, tissue biopsies, cells, including adipose cells or isolated blood cells, for example peripheral blood mononuclear cells, B cells, T cells and/or monocytes, and tissue samples, such as biopsy samples. Detecting an alteration in the amount of one or more of the proteins listed in Tables A, B, and/or C, using the methods disclosed herein indicates the prognosis or diagnosis of the subject, or indicates if a therapy is effective for treating a subject as described above. In some embodiments, the expression level of one or more of the proteins listed in Tables 6 and/or 7 is also assessed. Expression of proteins is the level of protein in a biological sample. Expression includes, but is not limited to, the production of the protein by translation of an mRNA and the half-life of the protein.

Any standard immunoassay format (such as ELISA, Western blot, or RIA assay) can be used to measure protein levels. Immunohistochemical techniques can also be utilized. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998), and Harlow & Lane, Antibodies, A Laboratory Manual, Cold Spring Harbor Publications, New York (1988); these references disclose a number of immunoassay formats and conditions that can be used to determine specific immunoreactivity. Generally, immunoassays include the use of one or more specific binding agents (such as antibodies) that specifically recognizes and can bind a molecule of interest, such a protein corresponding to a marker listed in Tables A, B, and/or C. Such binding agents can include a detectable label (such as a radiolabel, fluorophore or enzyme), that permits detection of the binding to the protein and determination of relative or absolute quantities of the molecule of interest in the sample. Although the details of the immunoassays may vary with the particular format employed, the method of detecting the protein in a sample generally includes the steps of contacting the sample with an antibody, which specifically binds to the protein under immunologically reactive conditions to form an immune complex between the antibody and the protein, and detecting the presence of and/or quantity of the immune complex (bound antibody), either directly or indirectly. The antibody can be a polyclonal or monoclonal antibody, or fragment thereof. In some examples, the antibody is a humanized antibody. In additional examples, the antibody is a chimeric antibody.

The antibodies can be labeled. Suitable detectable markers are described and known to the skilled artisan. For example, various enzymes, prosthetic groups, fluorescent materials, luminescent materials, magnetic agents, and radioactive materials can be used. Non-limiting examples of suitable enzymes include horseradish peroxidase, alkaline phosphatase, beta-galactosidase, or acetylcholinesterase. Non-limiting examples of suitable prosthetic group complexes include streptavidin/biotinm and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary magnetic agent is gadolinium, and non-limiting exemplary radioactive labels include ¹²⁵I, ¹³¹I, ³⁵S or ³H. Additional examples are disclosed above.

In another embodiment, the antibody that binds the protein of interest (the first antibody) is unlabeled and a second antibody or other molecule that can bind the antibody that binds the protein of interest is utilized. As is well known to one of skill in the art, a second antibody is chosen that is able to specifically bind the specific species and class of the first antibody. For example, if the first antibody is a mouse IgG, then the secondary antibody may be a goat anti-mouse-IgG. Other molecules that can bind to antibodies include, without limitation, Protein A and Protein G, both of which are available commercially.

Quantitation of proteins can be achieved by immunoassay. The amount of proteins can be assessed and optionally in a control sample. The amounts of protein in the sample from the subject of interest can be compared to levels of the protein found in samples form control subjects or to another control (such as a standard value or reference value). A significant increase or decrease in the amount can be evaluated using statistical methods known in the art.

In some non-limiting examples, a sandwich ELISA can be used to detect the presence or determine the amount of a protein in a sample. In this method, a solid surface is first coated with an antibody that specifically binds the protein of interest. The test sample containing the protein (such as, but not limited to, a blood, plasma, serum, or urine sample), is then added and the antigen is allowed to react with the bound antibody. Any unbound antigen is washed away. A known amount of enzyme-labeled protein-specific antibody is then allowed to react with the bound protein. Any excess unbound enzyme-linked antibody is washed away after the reaction. The substrate for the enzyme used in the assay is then added and the reaction between the substrate and the enzyme produces a color change. The amount of visual color change is a direct measurement of specific enzyme-conjugated bound antibody, and consequently the quantity of the protein present in the sample tested.

In an alternative example, a protein can be assayed in a biological sample by a competition immunoassay utilizing protein standards labeled with a detectable substance and an unlabeled antibody that specifically binds the protein of interest. In this assay, the biological sample (such as, but not limited to, a blood, plasma, serum, or urine sample), the labeled protein standards and the antibody that specifically binds the protein of interest are combined and the amount of labeled protein standard bound to the unlabeled antibody is determined. The amount of protein in the biological sample is inversely proportional to the amount of labeled protein standard bound to the antibody that specifically binds the protein of interest.

Mass spectrometry is particularly suited to the identification of proteins from biological samples, such those listed in Tables A, B, and C. Mass spectrometry also is particularly useful in the quantitation of peptides in a biological sample, for example using isotopically labeled peptide standards. The application of mass spectrometric techniques to identify proteins in biological samples is known in the art and is described, for example, in Akhilesh et al., Nature, 405:837-846, 2000; Dutt et al., Curr. Opin. Biotechnol., 11:176-179, 2000; Gygi et al., Curr. Opin. Chem. Biol., 4 (5): 489-94, 2000; Gygi et al., Anal. Chem., 72 (6): 1112-8, 2000; and Anderson et al., Curr. Opin. Biotechnol., 11:408-412, 2000.

Separation of ions according to their m/z ratio can be accomplished with any type of mass analyzer, including quadrupole mass analyzers (Q), time-of-flight (TOF) mass analyzers (for example, linear or reflecting) analyzers, magnetic sector mass analyzers, 3D and linear ion traps (IT), Fourier-transform ion cyclotron resonance (FT-ICR) analyzers, Orbitrap analyzers (like LTQ-Orbitrap LC/MS/MS), and combinations thereof (for example, a quadrupole-time-of-flight analyzer, or Q-TOF analyzer). A triple quadropole instrument can be used such as the Q-trap.

In some embodiments, the mass spectrometric technique is tandem mass spectrometry (MS/MS). Typically, in tandem mass spectrometry a protein gene product, such as those from Table A, B, and/or C, entering the tandem mass spectrometer is selected and subjected to collision induced dissociation (CID). The spectrum of the resulting fragment ion is recorded in the second stage of the mass spectrometry, as a so-called CID or ETD spectrum. Because the CID or ETD process usually causes fragmentation at peptide bonds and different amino acids for the most part yield peaks of different masses, a CID or ETD spectrum alone often provides enough information to determine the presence of a the protein of Tables A, B, or C. Suitable mass spectrometer systems for MS/MS include an ion fragmentor and one, two, or more mass spectrometers, such as those described above. Examples of suitable ion fragmentors include, but are not limited to, collision cells (in which ions are fragmented by causing them to collide with neutral gas molecules), photo dissociation cells (in which ions are fragmented by irradiating them with a beam of photons), and surface dissociation fragmentor (in which ions are fragmented by colliding them with a solid or a liquid surface). Suitable mass spectrometer systems can also include ion reflectors.

Prior to mass spectrometry, the sample can be subjected to one or more dimensions of chromatographic separation, for example, one or more dimensions of liquid or size exclusion chromatography. Representative examples of chromatographic separation include paper chromatography, thin layer chromatography (TLC), liquid chromatography, column chromatography, high performance liquid chromatography (HPLC), fast protein liquid chromatography (FPLC), ion exchange chromatography, size exclusion chromatography, affinity chromatography, high performance liquid chromatography (HPLC), nano-reverse phase liquid chromatography (nano-RPLC), polyacrylamide gel electrophoresis (PAGE), capillary electrophoresis (CE), reverse phase high performance liquid chromatography (RP-HPLC) or other suitable chromatographic techniques. Thus, in some embodiments, the mass spectrometric technique is directly or indirectly coupled with a one, two or three dimensional liquid chromatography technique, such as column chromatography, high performance liquid chromatography (HPLC or FPLC), reversed phase, ion exchange chromatography, size exclusion chromatography, affinity chromatography (such as protein or peptide affinity chromatography, immunoaffinity chromatography, lectin affinity chromatography, etc.), or one, two or three dimensional polyacrylamide gel electrophoresis (PAGE), or one or two dimensional capillary electrophoresis (CE) to further resolve the biological sample prior to mass spectrometric analysis.

A variety of mass spectrometry methods, including iTRAQ® and MRM, can be used. In some embodiments, quantitative spectroscopic methods, such as SELDI, are used to analyze protein expression in a sample. In one example, surface-enhanced laser desorption-ionization time-of-flight (SELDI-TOF) mass spectrometry is used to detect protein expression, for example by using the PROTEINCHIP™ (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption. Additional methods are disclosed in the examples section below.

Briefly, one version of SELDI uses a chromatographic surface with a chemistry that selectively captures analytes of interest, such as one or more proteins of interest. Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector.

In an additional example, the method may include detection of a protein of interest in a sample using an electrochemical immunoassay method. See, e.g., Yu et al., J. Am. Chem. Soc., 128:11199-11205, 2006; Mani et al., ACS Nano, 3:585-594, 2009; Malhotra et al., Anal. Chem., 82:3118-3123, 2010. In this method, an antibody that specifically binds the protein of interest is conjugated to terminally carboxylated single-wall carbon nanotubes (SWNT), multi-wall carbon nanotubes (MWCNT), or gold nanoparticles (AuNP), which are attached to a conductive surface. A sample (such as a blood, plasma or serum sample) is contacted with the SWNTs, MWCNTs, or AuNPs, and protein in the sample binds to the primary antibody. A second antibody conjugated directly or indirectly to a redox enzyme (such as horseradish peroxidase (HRP), cytochrome c, myoglobin, or glucose oxidase) binds to the primary antibody or to the protein (for example, in a “sandwich” assay). In some examples, the second antibody is conjugated to the enzyme. In other examples, the second antibody and the enzyme are both conjugated to a support (such as a magnetic bead). Signals are generated by adding enzyme substrate (e.g. hydrogen peroxide if the enzyme is HRP) to the solution bathing the sensor and measuring the current produced by the catalytic reduction.

In a particular example, the method includes a first antibody that specifically binds the protein of interest attached to an AuNP sensor surface. A sample (such as, but not limited to, a blood, plasma, serum, or urine sample) is contacted with the AuNP sensor including the first antibody. After the protein of interest binds to the first (capture) antibody (Ab1) on the electrode, a horseradish peroxidase (HRP)-labeled second antibody that specifically binds the protein of interest (HRP-Ab2) or beads conjugated to both a second antibody that binds the protein of interest and HRP are incubated with the sensor, allowing the second antibody to bind to the protein of interest. Biocatalytic electrochemical reduction produces a signal via reduction of peroxide activated enzyme following addition of hydrogen peroxide. Use of HRP is advantageous for arrays since immobilization of the electroactive enzyme label on the electrode eliminates electrochemical crosstalk between array elements, which can occur when detecting soluble electroactive product.

In some embodiments, iTRAQ® reagents are utilized. Multiple samples can be run simultaneously using different iTRAQ® reagents that label the individual samples with different mass identifiers. By way of example, sample one can be labeled with a mass identifier (or mass tag) that has a molecular weight of 114 amu, while sample two mass identifier (or mass tag) can have a molecular weight of 117. When the samples are combined and subjected to mass spectrometric analysis, a fragment peptide from sample two will have a predictable mass difference of three amu, compared to the same fragment peptide from sample one. In other words a peptide of identical sequence in sample one and sample two will be three amu heavier. This predictable mass difference can be used both to identify a peptide fragment (and hence the protein from which they were excised) and the relative quantities of each peptide in the samples.

In multiple reaction monitoring (MRM), tryptic peptides are used as markers for the abundance of specific proteins of interest, such as those listed in Tables A, B, and C. This selection is relatively straightforward if the protein has been identified by MS, such that the peptides are observable in a mass spectrometer (for example an LTQ Orbitrap). The process of establishing an MRM assay for a protein consists of a number of steps: 1) selection of the appropriate peptide(s) unique to the protein of interest and showing high MS signal response (prototypic peptides) which will help maximize the sensitivity of the assay; 2) selection of predominant peptide fragments specific (MS/MS) for the parent peptide (useful MRM transition); 3) for each peptide-fragment pair, optimization of specific MS parameters (for example, the collision energy) to maximize the signal response/sensitivity; 4) validation of the MRM assay to confirm peptide identity, for example by acquiring a full MS2 spectrum of the peptide in the triple quadrupole MS instrument used for MRM; 5) extraction of the final “coordinates” of the MRM assay, including the selected peptide and peptide fragments, the corresponding mass-to-charge ratios, the fragment intensity ratios, the associated collision energy, and the chromatographic elution time to be optionally used in time-constrained MRM analyses. In some examples, isotopically labeled internal peptide standards (with known concentrations determined by amino acid analysis) are used to facilitate absolute quantitation of selected peptides.

The concentration of the protein of interest, such as a protein corresponding to the markers listed in Tables A, B, and C, that is detected can be compared to a control, such as the concentration of the protein in a subject known not to have ASCVD, known not to have had an MI, or known not to be at risk for ASCVD and/or an MI. In other embodiments, the control is a standard value, such as a value that represents an average concentration of the protein of interest expected in a subject who does not have ASCVD and/or an MI, and/or is not at risk for ASCVD and/or an MI.

### Methods for Detection of mRNA

Gene expression can be evaluated by detecting mRNA encoding the gene of interest. Thus, the disclosed methods can include evaluating mRNA encoding one or more of the markers listed in Tables A, B, or C. Any of the methods disclosed above can utilize the detection of mRNA. In some embodiments, the disclosed methods also include evaluating mRNA encoding one or more of the markers listed in Tables 6 and/or Table 7.

RNA can be isolated from a sample from a subject, such as a biopsy, tissue sample, cardiac tissue, blood vessel, peripheral blood mononuclear cells, or isolated cells, such as white blood cells (B, T or mononuclear cells). RNA can also be isolated from a control, such as the same type of biological tissue from a healthy subject, for example a subject known not to have ASCVD or be at risk for an MI, using methods well known to one skilled in the art, including commercially available kits. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Biotechniques 6:56-60 (1988), and De Andres et al., Biotechniques 18:42-44 (1995). In one example, RNA isolation can be performed using purification kit, buffer set and protease from commercial manufacturers, such as QIAGEN® (Valencia, Calif.), according to the manufacturer's instructions. For example, total RNA from cells in culture (such as those obtained from a subject) can be isolated using QIAGEN® RNeasy® mini-columns. Other commercially available RNA isolation kits include MASTERPURE® Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from a biological sample can be isolated, for example, by cesium chloride density gradient centrifugation.

Methods of gene expression profiling include methods based on hybridization analysis of polynucleotides, methods based on sequencing of polynucleotides, and proteomics-based methods. In some examples, mRNA expression in a sample is quantified using Northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). In one example, RT-PCR can be used to compare mRNA levels in different samples, such as from subject that is undergoing treatment, to characterize patterns of gene expression, to discriminate between closely related mRNAs, and to analyze RNA structure.

Methods for quantitating mRNA are well known in the art. In some examples, the method utilizes RT-PCR. For example, extracted RNA can be reverse-transcribed using a GENEAMP® RNA PCR kit (Perkin Elmer, Calif., USA), following the manufacturer's instructions.

For example, TAQMAN® RT-PCR can be performed using commercially available equipment. The system can include a thermocycler, laser, charge-coupled device (CCD) camera, and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

To minimize errors and the effect of sample-to-sample variation, RT-PCR can be performed using an internal standard. The ideal internal standard is expressed at a constant level among different tissues, and is unaffected by an experimental treatment. RNAs commonly used to normalize patterns of gene expression are mRNAs for the housekeeping genes GAPDH, β-actin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Heid et al., Genome Research 6:986-994, 1996). Quantitative PCR is also described in U.S. Pat. No. 5,538,848. Related probes and quantitative amplification procedures are described in U.S. Pat. No. 5,716,784 and U.S. Pat. No. 5,723,591. Instruments for carrying out quantitative PCR in microtiter plates are available from PE Applied Biosystems (Foster City, Calif.).

The steps of a representative protocol for quantitating gene expression using fixed, paraffin-embedded tissues as the RNA source, including mRNA isolation, purification, primer extension and amplification are given in various published journal articles (see Godfrey et al., J. Mol. Diag. 2:84 91, 2000; Specht et al., Am. J. Pathol. 158:419-29, 2001). Briefly, a representative process starts with cutting about 10 μm thick sections of paraffin-embedded tissue samples or adjacent non-diseased tissue. The RNA is then extracted, and protein and DNA are removed. Alternatively, RNA is isolated directly from a tissue sample. After analysis of the RNA concentration, RNA repair and/or amplification steps can be included, if necessary, and RNA is reverse transcribed using gene specific promoters followed by RT-PCR.

The primers used for the amplification are selected so as to amplify a unique segment of the gene of interest (such as mRNA encoding one or more of the markers listed in Tables A, B, and C). In some embodiments, expression of other genes is also detected, such as the genes listed in Table 6 and Table 7. Primers that can be used to amplify mRNAs of interest are commercially available or can be designed and synthesized according to well-known methods.

An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In some examples, gene expression is identified or confirmed using the microarray technique. Thus, the expression profile can be measured in either fresh or paraffin-embedded tissue, using microarray technology. In this method, nucleic acid sequences of interest (including cDNAs and oligonucleotides) are plated, or arrayed, on a microchip substrate. The arrayed sequences are then hybridized with isolated nucleic acids (such as cDNA or mRNA) from cells or tissues of interest. Just as in the RT-PCR method, the source of mRNA typically is total RNA isolated from tissue or cells, and optionally from corresponding tissues or cells from a subject known not to be at risk for ASCVD and/or MI.

In a specific embodiment of the microarray technique, PCR amplified inserts of cDNA clones are applied to a substrate in a dense array. In some examples, the array includes probes specific to markers listed in Tables A, B, or C, or subsets of these markers. In some examples, probes specific for these nucleotide sequences are applied to the substrate, and the array can consist essentially of, or consist of these sequences. The microarrayed nucleic acids are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for genes of interest, such as those in Tables A, B, and C. Microarray analysis can be performed by commercially available equipment, following manufacturer's protocols, such as are supplied with Affymetrix GENECHIP® technology (Affymetrix, Santa Clara, Calif.), or Agilent's microarray technology (Agilent Technologies, Santa Clara, Calif.).

Serial analysis of gene expression (SAGE) is another method that allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag (see, for example, Velculescu et al., Science 270:484-7, 1995; and Velculescu et al., Cell 88:243-51, 1997).

In situ hybridization (ISH) is another method for detecting and comparing expression of genes of interest. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as one or more of the markers listed in Table A, B, and C. Sample cells or tissues are treated to increase their permeability to allow a probe to enter the cells. The probe is added to the treated cells, allowed to hybridize at pertinent temperature, and excess probe is washed away. A complementary probe is labeled so that the probe's location and quantity in the tissue can be determined, for example, using autoradiography, fluorescence microscopy or immunoassay. The sample may be any sample of interest.

In situ PCR is the PCR-based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products is generally achieved by one of two different techniques, indirect in situ PCR by ISH with PCR-product specific probes, or direct in situ PCR without ISH through direct detection of labeled nucleotides (such as digoxigenin-11-dUTP, fluorescein-dUTP, ³H-CTP or biotin-16-dUTP), which have been incorporated into the PCR products during thermal cycling.

In some embodiments of the detection methods, the expression of one or more “housekeeping” genes or “internal controls” can also be evaluated. These terms include any constitutively or globally expressed gene (or protein) whose presence enables an assessment of gene (or protein) levels. Such an assessment includes a determination of the overall constitutive level of gene transcription and a control for variations in RNA (or protein) recovery. The methods can also evaluate expression of other markers, such as one or more of the markers listed in Tables 6 and 7.

The concentration of the mRNA of interest, such as a mRNA corresponding to the markers listed in Tables A, B, and C, that is detected is compared to a control, such as the concentration of the mRNA in a subject known not to have ASCVD, known not to have had an MI, or known not to be at risk for ASCVD and/or an MI. In other embodiments, the control is a standard value, such as a value that represents an average concentration of the mRNA of interest expected in a subject who does not have ASCVD and/or an MI, and/or is not at risk for ASCVD and/or an MI.

### Arrays

In particular embodiments provided herein, arrays can be used to evaluate gene expression. When describing an array that consists essentially of probes or primers specific for the genes listed in Table A, Table B and/or Table C, such an array includes probes or primers specific for these genes, and can further include control probes (for example to confirm the incubation conditions are sufficient). In some examples, the array can consist essentially of probes or primers specific for CD5L, CLEC3B, and PON1, and optionally includes probes or primers specific for ORM1. In other examples, the array can consist essentially of probes or primers specific for CD5L and CRP, and optionally includes probes or primers specific for SERPINA10. In further examples, the array can consist essentially of probes or primers specific for MMRN2, CD5L, PPIA, CRP, COL18A1, AMY1A, and MCAM. The array can further include one or more control probes. In some examples, the array may further include additional, such as about 5, 10, 20, 30, 40, 50, 60, or 70 additional nucleic acids, such as those corresponding to the markers listed in Table 6 and/or Table 7. Exemplary control probes include GAPDH, β-actin, and 18S RNA. In one example, an array is a multi-well plate (e.g., 96 or 384 well plate). The oligonucleotide probes or primers can further include one or more detectable labels, to permit detection of hybridization signals between the probe and target sequence (such as those listed in Tables A, B, or C and/or Tables 6 and/or 7).

1. Array Substrates

The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfones, hydroxylated biaxially oriented polypropylene, aminated biaxially oriented polypropylene, thiolated biaxially oriented polypropylene, ethyleneacrylic acid, thylene methacrylic acid, and blends of copolymers thereof (see U.S. Pat. No. 5,985,567).

In general, suitable characteristics of the material that can be used to form the solid support surface include: being amenable to surface activation such that upon activation, the surface of the support is capable of covalently attaching a biomolecule such as an oligonucleotide thereto; amenability to “in situ” synthesis of biomolecules; being chemically inert such that at the areas on the support not occupied by the oligonucleotides or proteins (such as antibodies) are not amenable to non-specific binding, or when non-specific binding occurs, such materials can be readily removed from the surface without removing the oligonucleotides or proteins (such as antibodies).

In another example, a surface activated organic polymer is used as the solid support surface. One example of a surface activated organic polymer is a polypropylene material aminated via radio frequency plasma discharge. Other reactive groups can also be used, such as carboxylated, hydroxylated, thiolated, or active ester groups.

2. Array Formats

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In some examples, the array is a multi-well plate. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit low background fluorescence.

The array formats of the present disclosure can be included in a variety of different types of formats. A “format” includes any format to which the solid support can be affixed, such as microtiter plates (e.g., multi-well plates), test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as PCT applications WO 85/01051 and WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second set of rows that are now perpendicular to the first set. This process creates a multiple-channel array whose intersection generates a plurality of discrete cells.

The oligonucleotides can be bound to the polypropylene support by either the 3′ end of the oligonucleotide or by the 5′ end of the oligonucleotide. In one example, the oligonucleotides are bound to the solid support by the 3′ end. However, one of skill in the art can determine whether the use of the 3′ end or the 5′ end of the oligonucleotide is suitable for bonding to the solid support. In general, the internal complementarity of an oligonucleotide probe in the region of the 3′ end and the 5′ end determines binding to the support.

In particular examples, the oligonucleotide probes on the array include one or more labels, that permit detection of oligonucleotide probe:target sequence hybridization complexes.

### Kits

Kits are also provided. The kit can include probes, primers, or antibodies specific for the genes listed in Table A, Table B, and/or Table C, and can further include control probes, primers, and antibodies (for example to confirm the incubation conditions are sufficient). In some examples, the kit includes probes, primers and/or antibodies specific for CD5L, CLEC3B, and PON1, and optionally probes, primers and/or antibodies specific for ORM1. In other examples, the kit includes probes, primers and/or antibodies specific for CD5L and CRP, and optionally includes probes, primers and/or antibodies specific for SERPINA10. In further examples, the kit can include probes, primers and/or antibodies specific for MMRN2, CD5L, PPIA, CRP, COL18A1, AMY1A, and/or MCAM. The kit can further include one or more control probes, primers and/or antibodies

The kit can include a container and a label or package insert on or associated with the container. Suitable containers include, for example, bottles, vials, syringes, etc. The containers may be formed from a variety of materials such as glass or plastic. The container typically holds a composition including one or more of the probes, primers and/or antibodies. In several embodiments the container may have a sterile access port.

A label or package insert indicates that the composition is of use for evaluating if a subject is at risk for MI or ASCVD, or if a therapeutic agent is of use of the treatment of a subject. The label or package insert typically will further include instructions for use, such as particular assay conditions. The package insert typically includes instructions customarily included in commercial packages of products that contain information about the indications, usage, contraindications and/or warnings concerning the use of such products. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

The following examples are provided to illustrate certain particular features and/or embodiments. These examples should not be construed to limit the disclosure to the particular features or embodiments described.

### EXAMPLES

### Example 1

### Methods

Study Sample:

Framingham Heart Study (FHS) offspring cohort participants (n=5124) have undergone periodic clinic examinations approximately every four years since their enrollment in 1971 (Feinleib et al., Prev Med. 1975; 4:518-525). Onsite clinic examinations included medical history, questionnaires focused on cardiovascular disease (CVD) symptoms and risk factors, medication use, and lifestyle factors (Kannel et al., American journal of epidemiology. 1979; 110:281-290). During each clinic visit, a 12-lead electrocardiogram was obtained as well as measurements of blood pressure, height and weight, and collection of fasting blood specimens for glucose and lipoprotein measurements (Kannel W B, Wolf P A, Garrison R J. The Framingham study: An epidemiological investigation of cardiovascular disease. 1987; 112 edition). FHS offspring participants who attended examination 5 (1991-1995), 6 (1995-1998), 7 (1998-2001), or 8 (2005-2008) and were free of atherosclerotic cardiovascular disease (ASCVD) at that examination, were eligible. Participants at these examinations who developed a qualifying ASCVD event (see below) during follow up were selected as cases. Individuals who attended the same examination but remained free of ASCVD during the same follow-up period were eligible as controls. Among 3799 FHS offspring cohort participants who attended examination cycle 5, 3639 were free of prevalent ASCVD, and 338 developed incident ASCVD (cases) during follow up over a specified time period. ASCVD events included myocardial infarction (MI, n=136), death due to coronary heart disease (n=28), atherothrombotic brain infarction (n=70), coronary artery bypass grafting (n=57), and percutaneous transluminal coronary angioplasty (n=47). Clinical data and biological specimens for cases were selected from the FHS clinic examination visit immediately preceding ASCVD event occurrence (examination cycle 5 [n=85], 6 [n=66], 7 [n=150] or 8 [n=37]). For each case, one control was selected based on the following features: a) attended the same baseline examination cycle as the case; b) matched for age (±5 years), sex, smoking status, and statin use; c) free of ASCVD on the date of the event for the case. Clinical data and biological specimens for each control were selected from the same examination cycle as its matched case.

The Proteomics Pipeline:

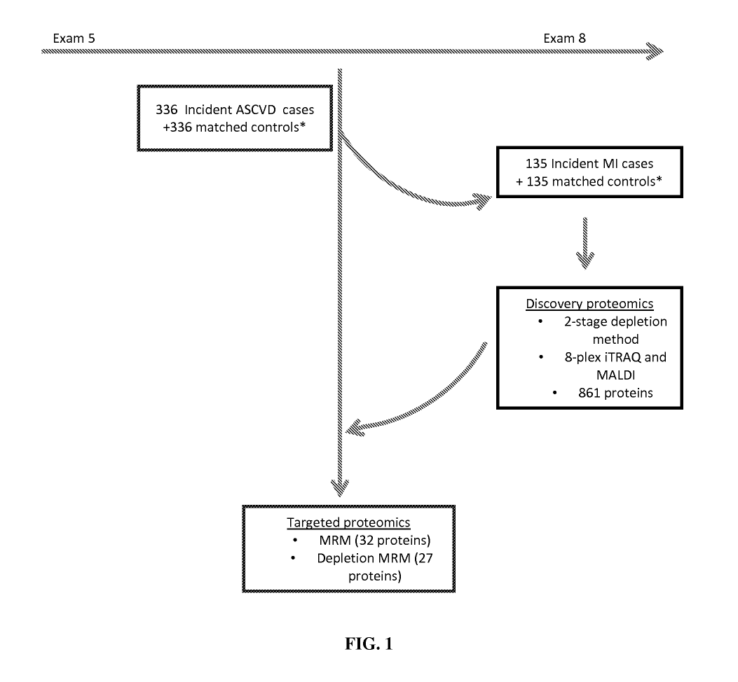

The experimental strategy was a discovery-validation pipeline (Rifai et al., Nat Biotechnol. 2006; 24:971-983; ubin DB. Multiple imputation for nonresponse in surveys. New York; Wiley; 1987) as illustrated in FIG. 1. In the discovery phase, iTRAQ® technology (Applied Biosystems, Foster City, Calif.) was utilized with multi-dimensional LC-MS/MS to analyze 136 myocardial infarction (MI) case-control pairs. Complete data collections was available for 135 case-control pairs. In the validation phase, the most promising protein biomarkers were chosen for multiple reaction monitoring (MRM) targeted measurements (32 by MRM and 27 by depletion MRM) based on a stepwise selection model. This targeted analysis was conducted on 338 qualifying ASCVD case-control pairs (676 samples total); proteomic data on 336 pairs was successfully completed.

Statistical Analyses:

(a) Single Marker Analysis:

All statistical analyses were performed using SAS software version 9.2 (Copyright, SAS Institute Inc., Cary, N.C., USA.). Blom's method was used to quantile-normalized values for each biomarker (Blom G. Statistical estimates and transformed beta-variables. New York: Wiley; 1958). Single marker and multiple marker analyses were performed for each analytic platform (iTRAQ® and MRM). Two analyses were performed for single markers: 1) paired t-test to compare means between cases and controls, and 2) conditional logistic regression (CLR) models with case status as the outcome, matched pairs as strata, normalized biomarker score as the main predictor, and adjusted for clinical covariates (systolic blood pressure, hypertension treatment status, total cholesterol, HDL cholesterol, diabetes status and BMI).

Multiple Marker Analysis:

Multiple marker analysis was performed using stepwise selection in conditional logistic regression models to identify a panel of markers (i.e. a multimarker) that was associated with case status. A threshold level of p=0.05 was used to enter and remain in stepwise selection. Because complete data was not available for many markers from the iTRAQ® platform (600 out of 861 protein markers had at least one missing value), multiple imputation (Rubin D B. Multiple imputation for nonresponse in surveys. New York: Wiley; 1987) was used to avoid sample size reduction due to missing values. A threshold of 60% completeness was set as an eligibility criterion for joint analysis. This resulted in 544 eligible markers (263 with partially missing values and 261 completely observed).

For the 544 eligible markers, markers we repeatedly and randomly shuffled into approximately 20 bins to generate imputed datasets, followed by stepwise selection within bins using Chen's method (Chen et al., Biometrics. 2011; 67:799-80). From each shuffle, a list of important markers was obtained (yielding p<0.05 in bin-specific final models). The frequency of selection across all rounds of randomizations was used as an importance scale, with markers selected more frequently deemed more important. In total, 260 shuffles were performed. Proteins chosen in at least 50% of stepwise selection models were considered key biomarkers. The list of key markers was short enough to enter into one imputation and stepwise selection model, allowing for selection of a final multimarker model. The detailed methods used for imputation is provided below.

For MRM and depletion MRM measurements, stepwise selection was performed in conditional logistic regression from all markers for association with MI (n=135 pairs) and with CVD (n=336 pairs).

Evaluation of Multimarker Panels:

The performance of the final multiple marker models was evaluated using C-statistics. With paired data, a C-statistic is defined as the proportion of pairs in which the case has a higher predicted probability of being a “case” than its matched control. Under the null hypothesis that the predictor is not associated with case status, the C-statistic is 0.50. The C-statistic was calculated for the model with clinical covariates only and the model with multiple protein biomarkers. The likelihood ratio test was also used to evaluate the significance of joint effect comparing the conditional logistic regression models with and without the panel of markers. In addition, among cases we calculated the gain in predicted probability of being a “case” from the clinical covariates model to the clinical covariates plus multiple protein biomarkers (i.e. multimarker) model.

### Example 2

### Proteomics Workflow

Briefly, a dual-stage protein depletion strategy was implemented to accommodate the quantitative analysis of the plasma proteome at a depth spanning nearly seven orders of circulating concentration (60 mg/mL-10 ng/mL) (Juhasz et al., J Proteome Res. 2010; 10:34-45).

Abundant protein depletion was implemented in two stages: an initial depletion of 14 selected abundant plasma proteins (5-mL, IgY14 column) followed by “Supermix” depletion (1-mL column, both from Sigma-Aldrich, St. Louis, Mo.) (Juhasz et al., supra). 100 μL of plasma aliquots were depleted in daily batches of eight samples. Proteins in the depletion flow-through were recovered and subsequently de-salted on a reversed-phase column. The protein samples were then reduced with tris (2-carboxyethyl)phosphine (TCEP) and alkylated with iodoacetamide. Following digestion with trypsin, each sample was labeled with a unique iTRAQ® tag. Peptide pools labeled with eight different iTRAQ® tags were then combined into an 8-plex experiment, referred to as an iTRAQ® mix. Two of the eight channels were reserved for reference samples that were created by pooling the primary samples. The remaining six channels represented primary samples. ITRAQ® tags producing the m/z 113 and m/z 117 reporter ions were assigned to the reference samples. The remaining tags (m/z 114, 115, 116, 118, 119, and 121 reporter ions) were randomized throughout all the iTRAQ® mixes to eliminate age, gender, and exam bias while ensuring that case-control pairs were assayed in the same iTRAQ® mix to maximize the precision of pair-wise comparisons. The peptide pool consisting of the entire ITRAQ® mix was then fractionated by strong cation exchange chromatography into nine fractions. Each fraction was further fractionated into 304 fractions by reversed-phase HPLC and directly spotted onto MALDI plates for MS/MS analysis using an AB/SCIEX 4800 TOF/TOF mass spectrometer (MDS SCIEX, Concord, ON,). Acquisition of LC-MS/MS data was optimized by algorithms to select and measure consistent sets of peptides from experiment to experiment (Juhasz et al., supra).

Relative quantification of peptides was carried out by determining relative intensities of reporter ions between the sample and (average of) reference channels.

Identification of peptides from the MS/MS spectra was achieved using the Mascot database searching tool (MatrixScience Ltd., London, UK) and BG-Medicine-based validation protocol to distinguish true and false positive peptide matches. This procedure provides false positive identification rates well below 1% if applied to sufficient number of experiments (Juhasz et al., supra). Relative quantification of proteins was achieved by assigning the median ratio from peptides mapped to the given protein. Normalization of protein expression data was carried out using a procedure described by Vandersompele et al. (Genome Biol. 2002; 3:RESEARCH0034.).

Target Analysis—

Multiple-Reaction Monitoring (MRM):_Qualification of marker candidates was performed through two passes of MRM analyses of 338 ASCVD case-control pairs. In the first pass plasma samples were processed without abundant protein depletion to measure the more abundant plasma proteins. The second pass targeted proteins at lower circulating levels through the MRM analysis of plasma samples following depletion of the 14 most abundant proteins. Designation of protein targets to the first or second pass MRM analysis was made based on abundance estimates from a collection of historical in-house plasma proteomics measurements.

Proteins from 10 μL neat or 30 μL depleted plasma aliquots were reduced and alkylated with tris(2-carboxyethyl)phosphine (TCEP) and iodoacetamide. Trypsin digestion was completed overnight at 1:10 enzyme:substrate ratio. Digestion was terminated by acidifying the digests with formic acid (to pH 2.5). Approximately 4 μg of peptide material (in 0.4 mg/mL solution) was analyzed by MRM (LC-MS/MS).

LC-MS/MS analyses were performed on a 4000QTrap and 5500QTrap (for Depletion MRM) linear ion trap instrument (AB/SCIEX, Concord, ON) interfaced with a U3000 HPLC system (Dionex, Sunnyvale, Calif.). The systems were plumbed to facilitate an in-line desalting step on a Poros R2 column with reverse flow. After desalting peptides were separated on a Targa C18 or Reprosil (for Depletion MRM) 150×1.0 mm column (Higgins Analytical and Dr. Maisch GmbH, respectively) utilizing a 200 μL/min flow rate. Peptide elution was carried out over a 21-min gradient from 2% Buffer B to 32% B (Buffer A: 5% acetonitrile, 0.1% formic acid, Buffer B: 95% acetonitrile, 0.1% formic acid). Following elution the HPLC columns were extensively washed with 95% B. The HPLC column compartment was kept at 50° C. during analysis.

For each target protein, two peptides were selected with two transitions (fragments) per peptide. Selection of these fragments was preceded by screening larger numbers of peptides and transitions (typically five peptides and five fragments for each fragment). Correct identity of peptides was confirmed by observing their correlation over a large number of individual samples.

Data collection was organized to acquisition batches sized to a 48-hr sample processing window. Randomization of acquisition order ensured the absence of age, gender, and exam number bias making sure the case-control pairs were analyzed in the same acquisition batch.

This experimental design normalizes peptide measurements to their mean measurement levels in the QC replicates. In this way, no isotope-labeled peptide standards were needed ensuring optimum multiplexing capacity of the LC-MS/MS runs at the expense of slightly increased measurement variability (Zhang et al., Mol Cell Proteomics. 2011; 10:M110 006593.). Before normalization, trend corrections were carried out if significant trends were detected in the series of QC samples. Normalization was performed by dividing the peak areas of individual transitions by the median of the same transition in the QC samples. In this manner, peptide quantities were reported as ratios, facilitating the conversion of peptide measurements into protein abundance measurements through simple averaging.

Multiple Imputation:

Through simulation an approach was studied for identifying important predictors in multivariable logistic models when missing values exist in hundreds of candidate variables. A subset of complete data and randomly masked values for some markers was used initially. Markers were shuffled into bins randomly, and multiple imputation was performed immediately, followed by stepwise selection. Rubin's rule was strictly implemented in stepwise selection process. Markers frequently chosen were deemed important. Choice of bin size, number of random shuffles of markers to bins, using prior information in MCMC imputation, and different importance thresholds for selecting important predictors were examined. The conclusion was that 26 was the best bin size among the values evaluated; using prior information in imputation not only improves convergence but also improves imputation quality; 200 shuffles is enough for a stable panel. Based on decisions from the masked-data explorations, the approach was applied to real data with 544 biomarkers on 135 myocardial infarction case-control pairs.

### Example 3

### iTRAQ® Discovery

Baseline characteristics of 135 pairs of myocardial infarction (MI) cases and controls with protein levels are summarized in Table 1. Systolic blood pressure, total cholesterol, and body mass index (BMI) were higher and HDL cholesterol was lower in cases than controls. Diabetes and hypertension treatment were more common in cases than controls. In total, 861 proteins were measured by iTRAQ®.

TABLE 1
Baseline characteristics of myocardial infarction cases and controls

| | MI Study | | ASCVD Study(MRM | |
|---|---|---|---|---|
| | (ITRAQ) | | and Depletion MRM) | |
| | Controls | Cases | Controls | Cases |
| Baseline characteristics | (n = 135) | (n = 135) | (n = 336) | (n = 336) |
| Age, years* | 65 ± 9 | 65 ± 9 | 65 ± 9 | 65 ± 9 |
| Female (%)* | 34 | 34 | 30 | 30 |
| Smoking, current (%)* | 24 | 24 | 17 | 17 |
| Statin use (%)* | 19 | 19 | 22 | 22 |
| Hypertension treatment | 37 | 47 | 35 | 49 |
| (%) | | | | |
| Diabetes, prevalent (%) | 7 | 28 | 9 | 27 |
| Lipid lowering therapy | 20 | 21 | 24 | 27 |
| (%) | | | | |
| Systolic blood pressure, | 131 ± 18 | 137 ± 18 | 131 ± 19 | 137 ± 20 |
| mm Hg | | | | |
| Total cholesterol, mg/Dl | 198 ± 34 | 205 ± 44 | 197 ± 33 | 203 ± 44 |
| HDL cholesterol, mg/Dl | 47 ± 15 | 45 ± 12 | 49 ± 15 | 45 ± 14 |
| BMI, kg/m² | 27.8 ± 4.4 | 28.8 ± 4.8 | 27.6 ± 4.4 | 29.0 ± 4.9 |

Presented are mean ± SD for continuous traits, or % for dichotomous traits
*Matching factors

### Example 2

### Single Marker Analysis

Among the iTRAQ® measured proteins, 34 had no known protein annotation (i.e. classified as unknown) and for 39 proteins multiple isoforms were identified; this left 753 unique known markers, among which results for 168 markers were available in fewer than 40 pairs of individuals. Markers were used with data available on at least 40 case-control pairs because for covariate adjusted analysis we required that at least 5 to 10 events per variable in a model were present (Peduzzi et al., J Clin Epidemiol. 1996; 49:1373-1379). Of 587 unique markers tested for association with MI, none had a p-value that attained overall significance after Bonferroni correction (0.05/587; p<8.5E-05). The top ten iTRAQ® proteins associated with MI (Table 2) were glycoprotein 5 (OR per 1 SD=0.44, 95% CI [0.27, 0.71]), CD5 antigen-like (0.55 [0.38, 0.79]), myoglobin (0.55 [0.37, 0.84]), inhibitor protein 1 (0.55 [0.36, 0.84]), C-reactive protein (1.75 [1.18, 2.59]), cyclophilin A (0.56 [0.37, 0.85]), contactin-1 (0.62 [0.45, 0.88]), albumin (0.43 [0.36, 0.85]), neural cell adhesion molecule 1 (0.61 [0.41,0.89]), and selenium-binding protein 1 (0.63 [0.42, 0.95]). Glycoprotein 5 was not associated with MI in the paired t-test (p=0.13) due to confounding by diabetes. Among diabetic subjects, the mean±standard deviation (SD) of glycoprotein 5 for cases and controls were −0.30±0.89 and 0.04±1.02; among subjects without diabetes, the mean±standard deviation (SD) of glycoprotein 5 for cases and controls were 0.46±1.00 and 0.71±0.54, respectively.

TABLE 2
Top protein biomarkers of myocardial infarction: results
of single marker analyses from iTRAQ mass spectrometry

| | | | Paired t Test | | Conditional Logistic | |
|---|---|---|---|---|---|---|
| | | | Mean difference | | Regression | |
| Gene | | # | (case-control) ± | P | Odds Ratio | P |
| Symbol | Protein Name | pairs | s.e. | value | (95% CI) | value |
| GP5 | Glycoprotein 5 | 120 | −0.16 ± 0.11 | 0.13 | 0.44(0.27, 0.71) | 0.0010 |
| CD5L | CD5 antigen-like | 135 | −0.18 ± 0.11 | 0.10 | 0.55(0.38, 0.79) | 0.0012 |
| MB | Myoglobin | 123 | −0.28 ± 0.10 | 0.0075 | 0.55(0.37, 0.84) | 0.0053 |
| YWHAZ | Protein kinase C | 124 | −0.2 ± 0.10 | 0.057 | 0.55(0.36, 0.84) | 0.0056 |
| | inhibitor protein 1 | | | | | |
| CRP | C-reactive protein | 129 | 0.35 ± 0.11 | 0.0016 | 1.75(1.18, 2.59) | 0.0058 |
| PPIA | Cyclophilin A | 135 | −0.26 ± 0.10 | 0.012 | 0.56(0.37, 0.85) | 0.0061 |
| CNTN1 | Contactin-1 | 135 | −0.20 ± 0.11 | 0.078 | 0.62(0.45, 0.88) | 0.0063 |
| ALB | Albumin | 135 | −0.22 ± 0.09 | 0.012 | 0.55(0.36, 0.85) | 0.0066 |
| NCAM1 | Neural cell adhesion | 135 | −0.31 ± 0.10 | 0.0020 | 0.61(0.41, 0.89) | 0.010 |
| | molecule 1 | | | | | |
| SELENBP1 | Selenium-binding | 106 | −0.32 ± 0.11 | 0.0067 | 0.63(0.42, 0.95) | 0.028 |
| | protein 1 | | | | | |
| GSN | Gelsolin | 135 | −0.30 ± 0.10 | 0.0032 | 0.75(0.53, 1.05) | 0.090 |
| MRC2 | Macrophage mannose | 109 | 0.26 ± 0.09 | 0.0068 | 1.51(0.93, 2.47) | 0.098 |
| | receptor 2 | | | | | |
| CLEC3B | Tetranectin | 135 | −0.26 ± 16 | 0.0046 | 0.77(0.56, 1.05) | 0.10 |
| SLC3A2 | 4F2 cell-surface | 135 | −0.23 ± 0.17 | 0.0054 | 0.79(0.56, 1.11) | 0.18 |
| | antigen heavy chain | | | | | |

This table shows markers with p-value < 0.01 in either conditional logistic regression or paired t-test. For each biomarker, data were rank normalized and have mean 0, SD 1. Differences, standard errors and odds ratios are in unit of one standard deviation. The conditional logistic regression model was adjusted for age, sex, current smoking status, statin use, systolic blood pressure, hypertension treatment status, total cholesterol, HDL cholesterol, diabetes status and BMI. Results are sorted by CLR values. All analyses are based on available data.

### Example 3

### Multiple Marker Analysis

To identify a multimarker protein panel from iTRAQ® that was significantly associated with MI, analyses were conducted for 544 proteins (measured in 60% or more of samples) using stepwise selection within bins as disclosed above. The number of times that a marker was retained in the final model varied from 1 to 259 (out of 260 runs).

Using multiple imputation-stepwise selection based on 26 protein markers that were selected >50% of time, a multimarker panel of 7 proteins associated with MI status was identified. The multimarker panel included cyclophilin A, CD5 antigen-like, cell surface glycoprotein MUC18, collagen alpha-1 (XVIII), salivary alpha-amylase 1, C-reactive protein, and multimerin-2 (Table 3).

TABLE 3
Protein biomarkers of myocardial infarction: results of
multimarker analyses from iTRAQ mass spectrometry

| | | | Final model | | |
|---|---|---|---|---|---|
| Gene | | Frequency | Odds | | |
| Symbol | Protein Name | selected* | Ratio | 95% CI | P value |
| PPIA | Cyclophilin A | 94% | 0.34 | (0.18, 0.63) | 0.0008 |
| CD5L | CD5 antigen-like | 99% | 0.48 | (0.29, 0.78) | 0.0040 |
| MCAM | Cell surface | 51% | 0.51 | (0.30, 0.86) | 0.013 |
| | glycoprotein | | | | |
| | MUC18 | | | | |
| COL18A1 | Collagen alpha- | 90% | 1.78 | (1.09, 2.89) | 0.021 |
| | 1(XVIII) chain | | | | |
| AMY1A | Alpha-amylase 1 | 98% | 0.54 | (0.32, 0.91) | 0.022 |
| | (salivary) | | | | |
| CRP | C-reactive protein | 96% | 1.87 | (1.09, 3.19) | 0.023 |
| MMRN2 | Multimerin-2 | 79% | 1.66 | (1.00, 2.75) | 0.049 |

These results were obtained through the multiple imputation procedure for missing values followed by stepwise selection in conditional logistic regression model, adjusting for age, sex, current smoking status, statin use, systolic blood pressure, hypertension treatment status, total cholesterol, HDL cholesterol, diabetes status and BMI. Results are sorted by final model p-value.
*Frequency selected in stage 1, across 260 shuffles.

The combination of these 7 protein biomarkers was associated with MI (likelihood ratio test chi-squared=53.7, df=7, p=2.6×10⁻⁹). The C-statistic from the model with clinical covariates only was 0.71. The median C-statistic for the multiple marker model (based on each of 20 imputed datasets) was 0.84 (confidence limits, 0.81 to 0.87). Among cases, the gain in predicted probability of being a case from the clinical model to multiple marker model had a mean of 0.16 (standard error 0.02). The two markers with largest effect on MI case status were cyclophilin A (OR per 1 SD=0.34, 95% CI [0.18, 0.63], p=0.0008) and CD5 antigen-like (0.48 [0.29, 0.79], p=0.0040). In single marker analysis (Table 2), glycoprotein 5 was the strongest predictor of MI. This marker was among the top 26 markers that were selected over 50% of the time, but it narrowly missed stepwise selection (p=0.056) adjusting for variables chosen in the final model.

### Example 4

### MRM and Depletion MRM

A. Single Marker Analysis

The top proteins in single- or multiple-marker analysis from iTRAQ® were assayed on 336 pairs of ASCVD cases and controls. Mass spectrometric multiple reaction monitoring (MRM) or depletion MRM was not attempted on any markers identified from iTRAQ® discovery if their plasma concentrations were below the detection limits of MRM technology (see Example 2). A total of 32 protein markers were measured by MRM and 27 by depletion MRM. In individual marker analyses, the top MRM markers associated with ASCVD were alpha-1-acid glycoprotein 1, C-reactive protein, ceruloplasmin, serum amyloid A-1 protein, gelsolin, tetranectin, hemopexin, paraoxonase 1, protein Z-dependent protease inhibitor, and leucine-rich alpha-2-glycoprotein (p<0.05 in the risk factor adjusted conditional logistic regression model; Table 4). The top proteins associated with ASCVD among those measured by depletion MRM included protein Z-dependent protease inhibitor and neural cell adhesion molecule 1 (Table 4).

TABLE 4
Top protein biomarkers of atherosclerotic cardiovascular disease: results of single
marker analyses from MRM and depletion MRM mass spectrometry

| | | | | Conditional Logistic | |
|---|---|---|---|---|---|
| | | Paired t test | | Regression | |
| Gene | | Mean difference | | Odds Ratio | |
| Symbol | Protein Name | (case-control) ± s.e. | P value | (95% CI) | P value |

MRM (n = 336 pairs)

| | | | | | |
|---|---|---|---|---|---|
| ORM1 | Alpha-1-acid | 0.29 ± 0.07 | <0.0001 | 1.43 (1.16, 1.76) | 0.0007 |
| | glycoprotein 1 | | | | |
| CRP | C-reactive protein | 0.35 ± 0.07 | <0.0001 | 1.38 (1.13, 1.69) | 0.0016 |
| CP | Ceruloplasmin | 0.15 ± 0.06 | 0.023 | 1.37 (1.11, 1.70) | 0.0037 |
| SAA1 | Serum amyloid A-1 | 0.27 ± 0.07 | 0.0003 | 1.30 (1.08, 1.57) | 0.0065 |
| | protein | | | | |
| GSN | Gelsolin | −0.30 ± 0.07 | <0.0001 | 0.77 (0.62, 0.95) | 0.013 |
| CLEC3B | Tetranectin | −0.30 ± 0.07 | <0.0001 | 0.76 (0.62, 0.95) | 0.015 |
| HPX | Hemopexin | 0.23 ± 0.07 | 0.0007 | 1.26 (1.03, 1.55) | 0.024 |
| PON1 | Paraoxonase 1 | −0.16 ± 0.07 | 0.017 | 0.79 (0.64, 0.98) | 0.031 |
| SERPINA10 | Protein Z-dependent | 0.20 ± 0.07 | 0.0031 | 1.25 (1.01, 1.53) | 0.037 |
| | protease inhibitor | | | | |
| LRG1 | Leucine-rich alpha-2- | 0.09 ± 0.07 | 0.17 | 1.24 (1.01, 1.52) | 0.039 |
| | glycoprotein | | | | |

Depletion MRM (n = 336 pairs)

| | | | | | |
|---|---|---|---|---|---|
| SERPINA10 | Protein Z-dependent | 0.20 ± 0.08 | 0.0070 | 1.21 (1.01, 1.46) | 0.036 |
| | protease inhibitor | | | | |
| NCAM1 | Neural cell adhesion | −0.19 ± 0.07 | 0.0067 | 0.82 (0.68, 1.00) | 0.048 |
| | molecule 1 | | | | |

This table shows markers with p-value <0.05 in conditional logistic regression (n = 336 pairs). For each biomarker, data were rank normalized and have mean 0, SD 1. Differences, standard errors and odds ratios are in unit of one standard deviation.
Conditional logistic regression results are adjusted for age, sex, current smoking status, statin use, systolic blood pressure, hypertension treatment status, total cholesterol, HDL cholesterol, diabetes status and BMI. Results are sorted by CLR values.

B. Multiple Marker Analysis

In risk factor adjusted multiple biomarker analysis from MRM and depletion MRM, alpha-1-acid glycoprotein 1 (OR per 1 SD=1.45, 95% CI [1.17,1.80], paraoxonase 1 (0.75 [0.60,0.94]), tetranectin (0.76 [0.60,0.95]), and CD5 antigen-like (0.81 [0.67,0.98]) jointly predicted ASCVD risk (Table 5). The likelihood ratio test showed that these proteins were associated with ASCVD (p<0.0001). The C-statistic of the model with clinical covariates only was 0.69; with the addition of the panel of four protein biomarkers, the C-statistic rose to 0.74. Among cases, the gain in predicted probability of being a case from the clinical model to multiple marker model had a mean of 0.032 (standard error 0.007).

TABLE 5
Protein biomarkers of atherosclerotic cardiovascular disease
and myocardial infarction: results of multimarker analyses
from MRM and depletion MRM mass spectrometry

| | | | |
|---|---|---|---|
| | | Odds Ratio | |
| Gene Symbol | Protein Name | (95% CI) | P value |

ASCVD (n = 336 pairs)

| | | | |
|---|---|---|---|
| ORM1 | Alpha-1-acid glycoprotein 1 | 1.45 (1.17, 1.80) | 0.0007 |
| PON1 | Paraoxonase 1 | 0.75 (0.60, 0.94) | 0.014 |
| CLEC3B | Tetranectin | 0.76 (0.61, 0.95) | 0.017 |
| CD5L | CD5 antigen-like | 0.81 (0.67, 0.98) | 0.031 |

MI (n = 135 pairs)

| | | | |
|---|---|---|---|
| SERPINA10 | Protein Z-dependent | 1.70 (1.16, 2.50) | 0.0070 |
| | protease inhibitor | | |
| CRP | C-reactive protein | 1.51 (1.03, 2.23) | 0.037 |
| CD5L | CD5 antigen-like | 0.70 (0.50, 0.98) | 0.039 |

Results are all from stepwise selection in conditional logistic regression models, adjusting for age, sex, current smoking status, statin use, systolic blood pressure, hypertension treatment status, total cholesterol, HDL cholesterol, diabetes status and BMI. Candidate markers include all 59 proteins measured by MRM and depletion MRM. P value of 0.05 was used as both enter and stay criteria. For each biomarker, data were rank normalized and have mean 0, SD 1. Odds ratios are in unit of one standard deviation.

Multiple marker analysis was repeated on the 135 MI case-control pairs included in the MRM and depletion MRM analysis. Protein Z-dependent protease inhibitor (OR per 1 SD=1.7, 95% CI [1.16, 2.50]), C-reactive protein (1.51 [1.03, 2.23]), and CD5 antigen-like (0.7 [0.50, 0.98]) in combination predicted the new onset of MI. The likelihood ratio test showed these protein biomarkers to be jointly associated with ASCVD (p<0.0001). The addition of these three markers to the clinical risk factors increased the C-statistic from 0.72 to 0.76. Among cases the mean gain in predicted probability of being a case from the clinical model to multiple marker model was 0.068 (standard error 0.015).

Through discovery and targeted proteomic studies, single protein biomarkers were identified that were associated with risk of MI or ASCVD. Panels of proteins were also identified that in aggregate improved MI and ASCVD risk prediction above and beyond established risk factors. Up to 587 protein biomarkers assayed by iTRAQ discovery mass spectrometry of MI cases and controls were included. After adjusting for established risk factors, the top iTRAQ derived protein biomarkers of MI in single marker analyses were glycoprotein 5, CD5 antigen-like, myoglobin, protein kinase C inhibitor protein 1, C-reactive protein, cyclophilin A, contactin-1, and albumin (p<0.01). Cyclophilin A, CD5 antigen-like, cell surface glycoprotein MUC18, collagen alpha-1(XVIII) chain, salivary alpha-amylase 1, C-reactive protein, and multimerin-2 emerged as a multimarker protein panel for MI (p=2.6×10⁻⁹). This panel of seven proteins improved MI risk prediction compared with clinical risk factors only, with a model C-statistic of 0.84 (versus 0.71 for the clinical risk factor only model). The improvement in prediction from the addition of the protein panel to the risk factor only model was of a magnitude that is consistent with clinical utility for the multimarker panel.

After obtaining these results, 32 and 27 proteins were targeted for measurement by MRM and depletion MRM mass spectrometry, respectively. Alpha-1-acid glycoprotein, C-reactive protein, ceruloplasmin, serum amyloid A-1 protein, gelsolin, tetranectin, hemopexin, paraoxonase 1, protein Z-dependent protease inhibitor, and leucine-rich-alpha-2-glycoprotein were the top MRM markers of ASCVD in single marker analyses (at p<0.05). The top proteins associated with ASCVD among those measured by depletion MRM were protein Z-dependent protease inhibitor and neural cell adhesion molecule 1 (at p<0.05). In multiple marker analysis of the combined MRM platforms, alpha-1-acid glycoprotein, paraoxonase 1, tetranectin, and CD5 antigen-like in combination predicted incident ASCVD (p<0.0001) and increased the C-statistic from the model with clinical covariates (0.69 to 0.73). The panel of three MRM markers that predicted MI (which included CD5 antigen-like, C-reactive protein, and protein Z-dependent protease inhibitor) increase the C-statistic from 0.72 to 0.76.

The proteomic work identified several novel biomarkers of MI and ASCVD; nine of these markers were significant in a prior proteomic study of cardiovascular disease related outcomes (Prentice et al., Genome Med. 2010; 2:48). Prentice et al. used proteomic screening of plasma from cases and controls in the Women's Health Initiative in an effort to identify proteomic biomarkers of coronary heart disease (CHD) and stroke. A difference between the Prentice et al. proteomics study and the study disclosed herein is that Prentice et al. pooled plasma samples from cases (and controls), precluding individual participant level analyses or complete adjustment for multiple ASCVD risk factors. Thus, 37 proteins were identified that were nominally associated with CHD and 47 proteins associated with stroke. In the Prentice et al. study, CD5 antigen-like, cyclophilin A, monocyte differentiation antigen CD14, multimerin-2, sulfhydryl oxidase 1, extracellular superoxide dismutase [Cu—Zn], apolipoprotein A-II, granulin, and insulin-like growth factor-binding protein 5 all had nominal p-values<0.05.

The MI prediction model utilized in the studies disclosed herein included novel proteins for which associations with ASCVD have not previously been reported at the population level. Such markers include: collagen alpha-1(XVIII) chain, cyclophilin A, CD5 antigen-like, and salivary alpha-amylase 1. Cyclophilin A (coded for by the gene PPIA) is a ubiquitous, intracellular protein that plays a role in protein folding and trafficking. It is secreted by cells in response to inflammatory stimuli, especially oxidative stress (Sato et al., Circulation. 2008; 117:3088-3098). Nigro et al. found that atherosclerosis was greater in APOE knockout mice vs. double knockouts for APOE and PPIA (Nigro et al., J Exp Med. 208:53-66).

CD5 antigen-like (CD5L) is a cell-surface ligand on activated lymphocytes (Biancone et al., J Exp Med. 1996; 184:811-819) and its function in atherogenesis largely remains unknown (Arai et al., Cell Metab. 2005; 1:201-213). Collagen alpha-1 (XVIII) chain (COL18A1) is a basement protein and its C-terminus encodes for endostatin, a 20 kDa proteolytic fragment that inhibits angiogenesis and atherosclerosis (O'Reilly et al., Cell. 1997; 88:277-285). When atherosclerosis prone APOE knockout mice were bred with COL18A1 knockout mice, there was extensive intimal neovascularization in the double knockouts (Moulton et al., Circulation. 2004; 110:1330-1336) because plaque neovascularization is thought to promote atherosclerosis, this may suggest a mechanistic relation between collagen alpha-1 (XVIII) and atherosclerosis. Salivary alpha-amylase 1 cleaves starch glycosidic linkages to produce smaller saccharides; alpha-amylase 1 is higher in populations that have evolved under high-starch diets and may modulate glycemic response after glucose intake (Santos et al., Nutrigenet Nutrigenomics. 2012; 5:117-131).

Among the proteins identified by MRM as being associated with ASCVD, alpha-1 acid glycoprotein is an acute phase protein that is secreted by the liver and measurable in plasma. It is an abundant plasma protein that increases in response to infection, inflammation, tissue injury, or cancer (Fournier et al., Biochim Biophys Acta. 2000; 1482:157-171; Hochepied et al., Cytokine Growth Factor Rev. 2003; 14:25-34). Lower circulating levels of paraoxonase 1 have been reported to be associated with risk of MI (see, for example, Ayub et al., Arterioscler Thromb Vasc Biol. 1999; 19:330-335) and in Framingham, an association was detected with similar directionality (OR per SD=0.79, 95% CI [0.64, 0.98], p=0.031).

Tetranectin is an adhesion molecule found on endothelial cells and platelets (Nielsen et al., Scand J Immunol. 1993; 37:39-42). Tetranectin is released by platelets and binds to the plasminogen kringle 4 domain; it enhances plasminogen activation and inhibits the proliferation of endothelial cells (Mougues et al., J Biomed Biotechnol. 2004; 2004:73-78), and decreased plasma tetranectin levels are associated with coronary artery disease. (Kamper et al., Ann Clin Biochem. 1998; 35 (Pt 3):400-407). The study disclosed herein used a prospective study design; higher tetranectin levels were inversely associated with risk of ASCVD (OR 0.76 [0.61, 0.95], p=0.017).

TABLE 6
Proteins measured by iTRAQ mass spectrometry associated with myocardial
infarction at p-value < 0.05 in single marker analyses

| | | | Paired t-test | | Conditional Logistic | |
|---|---|---|---|---|---|---|
| | | | Mean difference | | Regression | |
| Gene | | # | (case-control) ± | P- | Odds Ratio | P- |
| Symbol | Protein Name | pairs | s.e. | value | (95% CI) | value |
| TF | Transferrin | 135 | −0.19 ± 0.10 | 0.07 | 0.63 (0.44, 0.90) | 0.011 |
| ANGPTL3 | Angiopoietin-related | 109 | 0.26 ± 0.11 | 0.017 | 1.75 (1.13, 2.71) | 0.012 |
| | protein 3 | | | | | |
| SOD3 | Extracellular | 132 | −0.10 ± 0.11 | 0.36 | 0.63 (0.44, 0.91) | 0.013 |
| | superoxide | | | | | |
| | dismutase | | | | | |
| IGKC | Ig kappa chain C | 135 | −0.17 ± 0.10 | 0.10 | 0.62 (0.43, 0.91) | 0.013 |
| | region | | | | | |
| CDH13 | Cadherin-13 | 135 | −0.25 ± 0.10 | 0.010 | 0.61 (0.41, 0.91) | 0.014 |
| GAPDH | Glyceraldehyde- | 121 | −0.22 ± 0.12 | 0.06 | 0.63 (0.43, 0.91) | 0.014 |
| | 3-phosphate | | | | | |
| | dehydrogenase | | | | | |
| APOD | Apolipoprotein D | 135 | −0.23 ± 0.11 | 0.038 | 0.64 (0.45, 0.92) | 0.015 |
| CHGB | Secretogranin-1 | 93 | −0.22 ± 0.13 | 0.08 | 0.59 (0.38, 0.91) | 0.018 |
| AMY1A | Alpha-amylase 1 | 79 | −0.35 ± 0.15 | 0.019 | 0.58 (0.37, 0.91) | 0.019 |
| MCAM | Cell surface | 135 | −0.17 ± 0.11 | 0.12 | 0.66 (0.47, 0.93) | 0.019 |
| | glycoprotein MUC18 | | | | | |
| KRT10 | Keratin, type I | 100 | −0.23 ± 0.10 | 0.026 | 0.55 (0.33, 0.91) | 0.020 |
| | cytoskeletal 10 | | | | | |
| HSP90B1 | Endoplasmin | 83 | −0.18 ± 0.13 | 0.16 | 0.54 (0.32, 0.91) | 0.021 |
| FBN1 | Fibrillin-1 | 121 | −0.08 ± 0.11 | 0.46 | 0.64 (0.43, 0.93) | 0.021 |
| HSPG2 | Basement membrane- | 135 | −0.16 ± 0.12 | 0.18 | 0.70 (0.51, 0.95) | 0.023 |
| | specific heparan | | | | | |
| | sulfate proteoglycan | | | | | |
| | core protein | | | | | |
| IGHM | Ig mu chain C region | 135 | −0.15 ± 0.11 | 0.20 | 0.69 (0.50, 0.95) | 0.024 |
| FCRL5 | Fc receptor-like | 112 | −0.13 ± 0.11 | 0.23 | 0.63 (0.42, 0.94) | 0.024 |
| | protein 5 | | | | | |
| HSPA5 | 78 kDa | 135 | −0.20 ± 0.10 | 0.048 | 0.66 (0.46, 0.95) | 0.025 |
| | glucose-regulated | | | | | |
| | protein | | | | | |
| IGLC6 | Ig lambda-6 | 135 | −0.17 ± 0.11 | 0.13 | 0.67 (0.47, 0.95) | 0.026 |
| | chain C region | | | | | |
| LSAMP | Limbic system- | 94 | −0.14 ± 0.12 | 0.26 | 0.58 (0.36, 0.94) | 0.027 |
| | associated membrane | | | | | |
| | protein | | | | | |
| PIGR | Polymeric | 105 | −0.12 ± 0.10 | 0.24 | 0.57 (0.35, 0.94) | 0.028 |
| | immunoglobulin | | | | | |
| | receptor | | | | | |
| RTN4RL2 | Reticulon-4 | 124 | −0.11 ± 0.11 | 0.34 | 0.66 (0.46, 0.96) | 0.028 |
| | receptor-like 2 | | | | | |
| PNP | Purine nucleoside | 115 | −0.19 ± 0.12 | 0.12 | 0.65 (0.45, 0.95) | 0.028 |
| | phosphorylase | | | | | |
| NTM | Neurotrimin | 76 | −0.01 ± 0.03 | 0.77 | 0.60 (0.38, 0.95) | 0.028 |
| ALDOA | Fructose- | 132 | −0.25 ± 0.10 | 0.017 | 0.67 (0.47, 0.97) | 0.032 |
| | bisphosphate | | | | | |
| | aldolase A | | | | | |
| VNN1 | Pantetheinase | 135 | 0.27 ± 0.11 | 0.02 | 1.43 (1.03, 1.98) | 0.033 |
| PFN1 | Profilin-1 | 135 | −0.14 ± 0.10 | 0.19 | 0.68 (0.48, 0.97) | 0.033 |
| IL1RAP | Interleukin-1 | 135 | −0.27 ± 0.11 | 0.01 | 0.69 (0.48, 0.97) | 0.035 |
| | receptor accessory | | | | | |
| | protein | | | | | |
| COL18A1 | Collagen | 135 | 0.27 ± 0.11 | 0.011 | 1.45 (1.03, 2.06) | 0.036 |
| | alpha-1(XVIII) chain | | | | | |
| PDGFRB | Platelet-derived | 96 | −0.08 ± 0.12 | 0.48 | 0.59 (0.36, 0.97) | 0.037 |
| | growth factor | | | | | |
| | receptor beta | | | | | |
| PTPRS | Receptor-type | 125 | −0.13 ± 0.12 | 0.27 | 0.69 (0.49, 0.98) | 0.037 |
| | tyrosine-protein | | | | | |
| | phosphatase S | | | | | |
| IGJ | Immunoglobulin J | 129 | −0.13 ± 0.10 | 0.22 | 0.67 (0.46, 0.98) | 0.037 |
| | chain | | | | | |
| MMRN2 | Multimerin-2 | 135 | 0.08 ± 0.11 | 0.51 | 1.46 (1.02, 2.08) | 0.038 |
| COL1A2 | Collagen | 62 | −0.05 ± 0.03 | 0.13 | 0.60 (0.37, 0.98) | 0.040 |
| | alpha-2(I) chain | | | | | |
| MAN2A2 | Alpha-mannosidase 2x | 123 | −0.21 ± 0.10 | 0.036 | 0.68 (0.47, 0.98) | 0.040 |
| ADAMTS13 | A disintegrin and | 135 | −0.14 ± 0.10 | 0.16 | 0.68 (0.47, 0.98) | 0.040 |
| | metalloproteinase | | | | | |
| | with thrombospondin | | | | | |
| | motifs 13 | | | | | |
| GRN | Granulin | 103 | −0.27 ± 0.13 | 0.041 | 0.67 (0.46, 0.98) | 0.041 |
| SERPINA10 | Protein Z-dependent | 135 | 0.25 ± 0.10 | 0.014 | 1.45 (1.01, 2.08) | 0.041 |
| | protease inhibitor | | | | | |
| NID1 | Nidogen-1 | 112 | −0.07 ± 0.11 | 0.51 | 0.67 (0.45, 0.99) | 0.043 |
| SERPINE1 | Plasminogen activator | 100 | −0.05 ± 0.13 | 0.70 | 0.65 (0.43, 0.99) | 0.043 |
| | inhibitor 1 | | | | | |
| NOTCH3 | Neurogenic locus | 118 | −0.13 ± 0.12 | 0.25 | 0.69 (0.49, 0.99) | 0.043 |
| | notch homolog | | | | | |
| | protein 3 | | | | | |
| IGFBP6 | Insulin-like growth | 135 | −0.05 ± 0.10 | 0.64 | 0.68 (0.46, 0.99) | 0.044 |
| | factor-binding | | | | | |
| | protein 6 | | | | | |
| EPHA1 | Ephrin type-A | 132 | −0.04 ± 0.10 | 0.73 | 0.69 (0.48, 0.99) | 0.044 |
| | receptor 1 | | | | | |
| PDIA3 | Protein | 132 | −0.12 ± 0.11 | 0.25 | 0.70 (0.49, 0.99) | 0.046 |
| | disulfide-isomerase | | | | | |
| | A3 | | | | | |
| MMP2 | 72 kDa type IV | 135 | −0.15 ± 0.10 | 0.16 | 0.69 (0.48, 0.99) | 0.045 |
| | collagenase | | | | | |
| KRT6B | Keratin, type II | 98 | −0.17 ± 0.11 | 0.12 | 0.62 (0.39, 0.99) | 0.047 |
| | cytoskeletal 6B | | | | | |
| CST6 | Cystatin-M | 135 | −0.18 ± 0.11 | 0.090 | 0.70 (0.50, 1.00) | 0.048 |
| COL6A3 | Collagen | 135 | −0.11 ± 0.10 | 0.26 | 0.70 (0.50, 1.00) | 0.048 |
| | alpha-3(VI) chain | | | | | |
| S100A4 | Protein S100-A4 | 74 | −0.07 ± 0.03 | 0.030 | 0.56 (0.32, 1.00) | 0.050 |

TABLE 7
All proteins measured by MRM and depletion MRM except those in Table 4

| | | | | Conditional Logistic | |
|---|---|---|---|---|---|
| | | Paired t test | | Regression | |
| Gene | | Mean difference | P | Odds Ratio | P |
| Symbol | Protein Name | (case-control) ± s.e. | value | (95% CI) | value |

MRM

| | | | | | |
|---|---|---|---|---|---|
| CFI | CFI protein | 0.27 ± 0.07 | 0.0001 | 1.23 (1.00, 1.53) | 0.053 |
| APOA4 | Apolipoprotein A-IV | −0.05 ± 0.07 | 0.52 | 0.83 (0.68, 1.01) | 0.057 |
| SERPING1 | Plasma protease C1 inhibitor | 0.11 ± 0.07 | 0.13 | 1.21 (0.99, 1.47) | 0.067 |
| CFB | Complement factor B | 0.26 ± 0.07 | 0.0002 | 1.20 (0.99, 1.47) | 0.070 |
| CD5L | CD5 antigen-like | −0.12 ± 0.07 | 0.11 | 0.85 (0.71, 1.02) | 0.074 |
| C2 | Complement 2 | 0.22 ± 0.07 | 0.0012 | 1.20 (0.98, 1.46) | 0.075 |
| F13A1 | Coagulation factor XIII A | −0.12 ± 0.06 | 0.042 | 0.83 (0.65, 1.04) | 0.11 |
| | chain | | | | |
| HRG | Histidine-rich glycoprotein | 0.04 ± 0.07 | 0.58 | 0.85 (0.70, 1.04) | 0.11 |
| APOC3 | Apolipoprotein C-III | 0.12 ± 0.07 | 0.10 | 0.90 (0.74, 1.10) | 0.30 |
| PZP | Pregnancy zone protein | 0.08 ± 0.06 | 0.16 | 1.14 (0.89, 1.46) | 0.31 |
| APOD | PZP-like alpha-2- | −0.23 ± 0.07 | 0.0011 | 0.89 (0.69, 1.13) | 0.33 |
| | macroglobulin domain- | | | | |
| | containing protein 6 | | | | |
| ITIH1 | Apolipoprotein D | −0.01 ± 0.07 | 0.93 | 0.91 (0.74, 1.12) | 0.38 |
| CPN2 | Inter-alpha-trypsin inhibitor | 0.07 ± 0.07 | 0.31 | 1.09 (0.90, 1.31) | 0.39 |
| | heavy chain H1 | | | | |
| APOE | Carboxypeptidase N subunit 2 | 0.08 ± 0.07 | 0.27 | 0.91 (0.74, 1.13) | 0.39 |
| PROZ | Apolipoprotein E | 0.17 ± 0.07 | 0.0092 | 1.09 (0.88, 1.35) | 0.44 |
| ITIH2 | Vitamin K-dependent | −0.06 ± 0.07 | 0.38 | 0.93 (0.77, 1.13) | 0.48 |
| | protein Z | | | | |
| TF | Inter-alpha-trypsin inhibitor | 0.00 ± 0.07 | 0.95 | 0.94 (0.76, 1.15) | 0.52 |
| | heavy chain H2 | | | | |
| APOA2 | Transferrin | −0.17 ± 0.07 | 0.011 | 0.94 (0.74, 1.19) | 0.61 |
| APOA1 | Apolipoprotein A-II | −0.23 ± 0.07 | 0.0006 | 0.93 (0.67, 1.28) | 0.64 |
| F12 | AApolipoprotein-I | 0.05 ± 0.08 | 0.48 | 1.02 (0.85, 1.22) | 0.84 |

### Example 5

### Sequential Addition to a Multivariate Model

Conditional logistic regression adjusted for established clinical risk factors was performed. Protein biomarkers were added sequentially one at a time to determine the model performance and the incremental contribution of the model from the last added biomarker.

TABLE 8
iTRAQ Mass Spectrometry Protein Biomarkers of Myocardial Infarction (MI) when
Sequentially Added to a Multivariable Model with Clinical Risk Factors

| | Protein markers | | | Chi-square | | |
|---|---|---|---|---|---|---|
| | included in | | Overall | change from | | |
| | the model | Overall | model | previous | Model SBC | |
| iTRAQ proteins | (newly added | P value for | Chi- | model | (lower is | c-statistic |
| to predict MI | marker in bold) | markers added | square | (p-value) | better) | of model |
| Clinical risk | NA | NA | 38.7 | NA | 182.0 | 0.711 |
| factors only | | | | | | |
| Risk factors | CD5L | 0.0008 | 50.7 | 12.0 (0.00053) | 181.3 | 0.733 |
| plus CD5L | | | | | | |
| Risk factors | CD5L PPIA | 0.00009 | 58.2 | 7.5 (0.0062) | 179.3 | 0.748 |
| plus CD5L PPIA | | | | | | |
| Risk factors | CD5L PPIA | 4.4e−6 | 67.2 | 8.9 (0.0027) | 176.0 | 0.770 |
| plus CD5L PPIA | CRP | | | | | |
| CRP | | | | | | |
| Risk factors | CD5L PPIA | 3.8e−7 | 75.0 | 7.8 (0.0052) | 173.7 | 0.815 |
| plus CD5L PPIA | CRP COL18A1 | | | | | |
| CRP COL18A1 | | | | | | |
| Risk factors | CD5L PPIA | 1.4e−8 | 84.7 | 9.7 (0.0018) | 169.7 | 0.837 |
| plus CD5L PPIA | CRP COL18A1 | | | | | |
| CRP COL18A1 | AMY1A | | | | | |
| AMY1A | | | | | | |
| Risk factors | CD5L PPIA | 1.9e−9 | 91.5 | 6.8 (0.0091) | 168.4 | 0.837 |
| plus CD5L PPIA | CRP COL18A1 | | | | | |
| CRP COL18A1 | AMY1A | | | | | |
| AMY1A MCAM | MCAM | | | | | |
| Risk factors | CD5L PPIA | 9.7e−10 | 95.5 | 4.0 (0.045) | 170.0 | 0.844 |
| plus CD5L PPIA | CRP COL18A1 | | | | | |
| CRP COL18A1 | AMY1A | | | | | |
| AMY1A MCAM | MCAM | | | | | |
| MMRN2 | MMRN2 | | | | | |

TABLE 9
MRM/Depletion MRM Mass Spectrometry Protein Biomarkers of Atherosclerotic Cardiovascular Disease
(ASCVD) when Sequentially Added to a Multivariable Model with Clinical Risk Factors

| | Protein markers | | | Chi-square | | |
|---|---|---|---|---|---|---|
| | included in | | Overall | change from | | |
| | the model | Overall model | model | previous | Model SBC | |
| MRM proteins to | (newly added | P value when | Chi- | model | (lower is | c-statistic |
| predict ASCVD | marker in bold) | marker added | square | (p-value) | better) | of model |
| Clinical risk | NA | NA | 80.9 | NA | 426.2 | 0.694 |
| factors only | | | | | | |
| Risk factors | ORM1 | 0.0007 | 92.5 | 11.5 (0.0007) | 421.1 | 0.730 |
| plus ORM1 | | | | | | |
| Risk factors | ORM1 | 0.0002 | 97.7 | 5.2 (0.023) | 422.4 | 0.721 |
| plus ORM1 and | CLEC3B | | | | | |
| CLEC3B | | | | | | |
| Risk factors | ORMI CLEC3B | 7.6e−5 | 102.6 | 4.9 (0.027) | 424.1 | 0.733 |
| plus ORMI, | PON1 | | | | | |
| CLEC3B and | | | | | | |
| PON1 | | | | | | |
| Risk factors | ORMI CLEC3B | 2.2e−5 | 107.6 | 5.0 (0.025) | 425.5 | 0.738 |
| plus ORMI, | PON1 | | | | | |
| CLEC3B, PON1 | CD5L | | | | | |
| and CD5L | | | | | | |

TABLE 10
Protein biomarkers of myocardial infarction: results of multimarker
analyses from iTRAQ mass spectrometry

| | | Final model | | | |
|---|---|---|---|---|---|
| | | Odds | | | Frequency |
| Marker | Protein Name | Ratio | 95% CI | P value | selected |
| PPIA | Cyclophilin A | 0.34 | (0.18, 0.63) | 0.0008 | 94% |
| CD5L | CD5 antigen- | 0.48 | (0.29, 0.78) | 0.0040 | 99% |
| | like | | | | |
| MCAM | Cell surface | 0.51 | (0.30, 0.86) | 0.013 | 51% |
| | glycoprotein | | | | |
| | MUC18 | | | | |
| COL18A1 | Collagen alpha- | 1.78 | (1.09, 2.89) | 0.021 | 90% |
| | 1(XVIII) chain | | | | |
| AMY1A | Alpha-amylase | 0.54 | (0.32, 0.91) | 0.022 | 98% |
| | 1 (salivary) | | | | |
| CRP | C-reactive | 1.87 | (1.09, 3.19) | 0.023 | 96% |
| | protein | | | | |
| MMRN2 | Multimerin-2 | 1.66 | (1.00, 2.75) | 0.049 | 79% |

Results are from conditional logistic regression model, adjusting for age, sex, current smoking status, statin use, systolic blood pressure, hypertension treatment status, total cholesterol, HDL cholesterol, diabetes status and BMI. Results are sorted by final model p-value.

### Example 6

### Additional Information

TABLE 11
ASCVD Markers

| | | Expression level is |
|---|---|---|
| | Expression level is increased in | decreased in biological |
| Marker | biological sample compared to | sample compared to |
| (ASCVD) | control | control |
| CD5L | | X |
| CLEC3B | | X |
| POIN1 | | X |
| ORM1 | X | |

TABLE 12
MI Markers

| | Expression level is increased | Expression level is |
|---|---|---|
| | in biological sample | decreased in biological |
| Marker (MI) | compared to control | sample compared to control |
| CD5L | | X |
| CRP | X | |
| SERPINA10 | X | |

TABLE 13
MI Markers

| | Expression level is increased | Expression level is decreased |
|---|---|---|
| Marker | in biological sample | in biological sample |
| (MI) | compared to control | compared to control |
| MMRN2 | X | |
| CD5L | | X |
| PPIA | | X |
| CRP | X | |
| COL18A1 | X | |
| AMY1A | | X |
| MCAM | | X |

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. (canceled)

2. A method of detecting or determining the likelihood that a subject will develop atherosclerotic cardiovascular disease, comprising:
obtaining a biological sample from the subject, wherein the biological sample is a blood, tissue, plasma or serum sample; and
performing an assay that detects a level of alpha-1-acid glycoprotein 1 (ORM1), in the biological sample.

3. (canceled)

4. A method of determining if a pharmaceutical agent is effective for treatment or prevention of atherosclerotic cardiovascular disease in a subject, comprising:
obtaining a biological sample from the subject, wherein the biological sample is a blood, tissue, plasma or serum sample from a human subject; and
performing an assay that detects a level of alpha-1-acid glycoprotein 1 (ORM1) in the biological sample from the subject.

5. (canceled)

6. (canceled)

7. (canceled)

8. The method of claim 2, further comprising assessing one or more Framingham risk factors for the subject.

9. The method of claim 2, wherein the assay that detects the level of ORM1 detect detects ORM1 mRNA.

10. The method of claim 9, wherein the assay is polymerase chain reaction, a microarray analysis or a hybridization reaction.

11. (canceled)

12. The method of claim 2, wherein the assay that detects the level of ORM1 detects ORM1 protein.

13. The method of claim 12, wherein the assay is mass spectrometry; and
diagnosing the subject with atherosclerosis or determining that it is likely that the subject will develop atherosclerosis when the presence of ORM1 is detected by mass spectrometry.

14. The method of claim 13, wherein the mass spectrometry is MALDI-TOF mass spectrometry and/or LC-mass spectrometry.

15. The method of claim 12, wherein performing the assay that detects ORM1 protein comprises contacting the biological sample or a component thereof with an antibody that specifically binds ORM1 protein;
detecting binding between the ORM1 in the sample and the antibody; and
diagnosing the subject with atherosclerosis or determining that it is likely that the subject will develop atherosclerosis when the presence of ORM1 is detected in the biological sample by binding to the antibody.

16. (canceled)

17. The method of claim 15, wherein the assay is a Western blot, an enzyme linked immunosorbent assay, or a radioimmunoassay.

18. The method of claim 15, wherein the antibody that specifically binds ORM1 protein is directly labeled.

19. The method of claim 18, wherein the label is a radioactive marker, a fluorescent marker, an enzyme or a metal.

20. A method of diagnosing and treating atherosclerotic cardiovascular disease in a subject, comprising
obtaining a biological sample from the subject, wherein the biological sample is a blood, tissue, plasma or serum sample;
detecting whether alpha-1-acid glycoprotein 1 (ORM1) is present in the biological sample;
diagnosing the subject with atherosclerotic cardiovascular disease when the presence of ORM1 is detected in the biological sample; and
administering to the subject a therapeutically effective amount of an agent for the treatment of atherosclerotic cardiovascular disease if the subject is determined to have atherosclerotic cardiovascular disease.

21. The method of claim 20, wherein the agent is a statin, niacin, a fibrate, a bile acid binding resin, a cholesterol absorption inhibitor, a PCSK9-targeting drug, an LDL-targeting drug or an HDL-targeting drug.

22. The method of claim 2, wherein the method does not comprise measuring lipoprotein.

23. The method of claim 22, wherein the method does not comprise measuring high density lipoproteins or determining a high density lipoprotein subpopulation.

24. The method of claim 2, wherein the method does not comprise measuring inflammatory markers.

25. The method of claim 24, wherein the inflammatory marker is C reactive protein.

26-70. (canceled)

71. The method of claim 20, wherein the assay that detects the level of ORM1 detects ORM1 protein.

72. The method of claim 71, wherein the assay is MALDI-TOF mass spectrometry and/or LC-mass spectrometry, and diagnosing the subject with atherosclerosis or determining that it is likely that the subject will develop atherosclerosis when the presence of ORM1 is detected by MALDI-TOF mass spectrometry or LC-mass spectrometry.

73. The method of claim 71, wherein performing the assay that detects ORM1 protein comprises
contacting the biological sample or a component thereof with an antibody that specifically binds ORM1 protein;
detecting binding between the ORM1 in the sample and the antibody; and
diagnosing the subject with atherosclerosis or determining that it is likely that the subject will develop atherosclerosis when the presence of ORM1 is detected in the biological sample by binding to the antibody.
